(19) United States
(12) Patent Application Publication
(10) Pub. No.: US 2007/0212716 A1
(43) Pub. Date: Sep. 13, 2007

(54) Nezara viridula Ecdysone Receptor

(75) Inventors: Donya Tohidi-Esfahani, Hornsby (AU); Gary Noel Hannan, Hunters Hill (AU); Lloyd Douglas Graham, North Ryde (AU); Ronald Johnston Hill, Forestville (AU)

Correspondence Address: GREENLEE WINNER AND SULLIVAN P C, 4875 PEARL EAST CIRCLE, SUITE 200, BOULDER, CO, 80301, US

(73) Assignee: Commonwealth Scientific & Industrial Research Org., Clayton South (AU)

(21) Appl. No.: 11/673,456

(22) Filed: Feb. 9, 2007

(30) Foreign Application Priority Data
Feb. 10, 2006 (AU) 2006900641

(51) Int. Cl. C12Q 1/68; G01N 33/53; C07H 21/04; C12P 21/06; C12N 5/06; C07K 14/705

(52) U.S. Cl. 435/6; 435/7.1; 435/69.1; 435/348; 530/350; 536/23.5

(57) ABSTRACT

Provided are protein and coding sequences for the ecdysone receptor polypeptide isoforms and ultraspiracle polypeptide from Nezara viridula and methods of employing them.

### CROSS REFERENCE TO RELATED APPLICATIONS

This application claims the benefit of Australian Provisional Patent Application No. 200690064,1 filed 10 Feb. 2006, which is incorporated herein by reference in its entirety.

### FIELD OF THE INVENTION

The present invention relates to the identification and characterisation of EcR polypeptide isoforms and an ultraspiracle (USP) polypeptide from the Nezara viridula (the Green Vegetable Bug) ecdysone receptor, and the polynucleotides encoding therefor. The present invention also relates to the use of such receptors, and such polypeptides in screening methods, particularly in identifying modulating agents of such receptors.

### BACKGROUND TO THE INVENTION

The green vegetable bug, Nezara viridula, is distributed throughout America, in various Pacific Islands, Africa, Asia, New Zealand, and Australia (QLD, NSW, VIC, NT and TAS). There are indications that geographical distribution is spreading owing to climate change (Barclay, 2004). The bug is a significant pest on a number of crops of economic importance including rice, cotton, beans, cabbage, potatoes, mango, tomatoes, citrus fruits, nuts (e.g. macadamia, pecan).

Chemical agents currently used in the control of Nezara viridula include carbamates and organophosphates. Therefore, there exists a need for providing safer and more effective agents for controlling such pests.

Novel compounds mimicking insect hormones act by complexing with insect members of the steroid receptor superfamily to control insect development and are likely candidates for pesticides with desirable properties. In recent years the characterisation of insect steroid receptors, that play a critical role in insect development, has provided targets and molecular tools for the discovery of novel chemistries for use in the constant fight against insect pests. Most of the initial research carried out on the molecular biology of the insect steroid receptor superfamily has been on Drosophila melanogaster (Diptera).

Growth, molting, and development in insects are generally regulated by the ecdysteroid hormone, 20-hydroxyecdysone (2β, 3β, 14α, 20R,22R,25-hexahydroxy-5β-cholest-7-′ene-6-one), hereafter referred to as “ecdysone” (molting hormone) and the juvenile hormones (Dhadialla et al., 1998). The molecular target for ecdysone in insects, the ecdysone receptor, consists of at least the ecdysone receptor protein (EcR) and the ultraspiracle protein (USP). EcR and USP are members of the nuclear steroid receptor super family that is characterized by signature DNA and ligand binding domains, and a transcription activation domain (Koelle et al., 1991). Ecdysone receptors are responsive to a number of steroidal compounds such as ecdysone, ponasterone A and muristerone A. Recently, non-steroidal compounds with ecdysteroid agonist activity have been described, including the commercially available insecticides tebufenozide and methoxyfenozide (see International Patent Application No. PCT/EP96/00686 and U.S. Pat. No. 5,530,028). Both analogs have exceptional safety profiles for non-target organisms.

Polynucleotides encoding EcR proteins have been cloned from a variety of insect species, including Dipterans (see U.S. Pat. Nos. 5,514,578 and 6,245,531 B1), Lepidopterans, Orthopterans, Hemipterans, and one Homopteran Aphid, all from the class Arthropod, a fruit fly Drosophila melanogaster EcR (“DmEcR”; Koelle et al., 1991), a yellow fever mosquito Aedes aegypti EcR (“AaEcR”; Cho et al., 1995), a blowfly Lucilia capitata (“LcEcR”), a sheep blowfly Lucilia cuprina EcR (“LucEcR”; Hannan and Hill, 1997), a blowfly Calliphora vicinia EcR (“CvEcR”), a Mediterranean fruit fly Ceratitis capitata EcR (“CcEcR”; Verras et al., 1999), a locust Locusta migratoria EcR (“LmEcR”; Saleh et al., 1998), an aphid Myzus persicae EcR (“MpEcR” and International Patent Application Publication WO99/36520). The nucleotide and/or amino acid sequences of these ecdysone receptors have been determined and are publicly available.

The ecdysone receptor complex typically includes proteins that are members of the nuclear receptor superfamily wherein all members are generally characterized by the presence of an amino-terminal transactivation domain, a DNA binding domain, and a ligand binding domain separated from the DNA binding domain by a hinge region. The DNA binding domain is characterized by the presence of two cysteine zinc fingers between which are two amino acid motifs, the P-box and the D-box, which confer specificity for ecdysone response elements. These domains may be either native, modified, or chimeras of different domains of heterologous receptor proteins. The EcR and USP proteins, as subsets of the steroid receptor family, also possesses less well-defined regions responsible for heterodimerization properties. Because the domains of nuclear receptors are modular in nature, the ligand binding domain, DNA binding domain, and transactivation domains may be interchanged between receptors to produce functional chimeric receptors with new properties.

The insect ecdysone receptor protein (EcR) heterodimerizes with the ultraspiracle protein (USP), the insect homologue of the mammalian RXR, to form the ecdysone receptor which binds ecdysteroids and ecdysone receptor response elements and activates transcription of ecdysone responsive genes (Riddiford et al., 2000).

The EcR/USP/ligand complexes play important roles during insect development and reproduction. The EcR and USP proteins are members of the steroid hormone receptor superfamily and as such have five modular domains, A/B (transactivation), C (DNA binding, heterodimerization), D (hinge, heterodimerization), E (ligand binding, heterodimerization and transactivation and in some cases, F (transactivation), domains. Some of these domains such as A/B, C and E retain their function when they are fused to other proteins.

The present inventors have now isolated and characterised two novel EcR proteins and a novel USP protein from N. viridula. Novel polypeptides and polynucleotides encoding these proteins are provided. In targeting such ecdysone receptors of N. viridula the inventors have identified useful methods for identifying new modulating agents of these receptors that would serve as effective agents to control pest insects.

### SUMMARY OF THE INVENTION

The present inventors have successfully characterised two novel EcR isoforms and a novel ultraspiracle (USP) from N. viridula. The first novel EcR isoform, NvEcR10, has the polynucleotide sequence of SEQ ID No: 1 and the amino acid sequence of SEQ ID No:4. The second novel EcR isoform, NvEcR11, has the polynucleotide sequence of SEQ ID No:2 and the amino acid sequence of SEQ ID No:5. The present results indicate that the two NvEcR isoforms isolated differ in their E domains, a region of the molecule not normally affected by differential splicing. A novel ultraspiracle (USP) from N. viridula has also been identified as having the polynucleotide sequence of SEQ ID No:3 and the amino acid sequence of SEQ ID No:6. In addition, the present invention provides the means for identifying and developing specific modulating agents, such as ligands which bind and either agonise or antagonise the ecdysone receptors, thereby functioning as highly-specific pesticides against N. viridula, offering significant commercial and environmental benefits.

One aspect of the present invention provides an isolated polynucleotide comprising a nucleotide sequence which encodes or is complementary to a sequence which encodes an EcR polypeptide of an N. viridula ecdysone receptor, wherein the polynucleotide comprises a nucleic acid sequence at least 60%, preferably at least 80% identical, more preferably at least 90%, 95%, 96%, 97%, 98% or 99% identical to the sequence set forth in SEQ ID No:1 or SEQ ID No:2 and most preferably, the sequence set forth in SEQ ID No:1 or SEQ ID No:2.

In a second aspect, the present invention provides an isolated polynucleotide comprising a nucleotide sequence which encodes or is complementary to a sequence which encodes a USP polypeptide of a N. viridula ecdysone receptor, wherein the polynucleotide comprises a nucleic acid sequence at least 60%, preferably at least 80% identical, more preferably at least 90%, 95%, 96%, 97%, 98% or 99% identical to the sequence set forth in SEQ ID No:3, and most preferably, the sequence set forth in SEQ ID No:3.

In a third aspect, the present invention provides an isolated polynucleotide comprising a nucleotide sequence which encodes or is complementary to a sequence which encodes an EcR polypeptide of an N. viridula ecdysone receptor, the polypeptide comprising an amino acid sequence at least 60%, preferably at least 80% identical, more preferably at least 90%, 95%, 96%, 97%, 98% or 99% identical to the sequence set forth in SEQ ID No:4 or SEQ ID No:5 and most preferably, the amino acid sequence set forth in SEQ ID No:4 or SEQ ID No:5.

In a fourth aspect, the present invention provides an isolated polynucleotide comprising a nucleotide sequence which encodes or is complementary to a sequence which encodes a USP polypeptide of a N. viridula ecdysone receptor, the polypeptide comprising an amino acid sequence at least 60%, preferably at least 80% identical, more preferably at least 90%, 95%, 96%, 97%, 98% or 99% identical to the sequence set forth in SEQ ID No:6 and most preferably, the amino acid sequence set forth in SEQ ID No:6.

In a fifth aspect, the present invention provides an isolated polynucleotide comprising a sequence that is at least 10 nucleotides in length capable of hybridising under at least high stringency conditions to the nucleotide sequence set forth in SEQ ID No:1, SEQ ID No:2 or SEQ ID No:3; or to a complementary nucleotide sequence thereto, wherein high stringency conditions are a hybridisation and/or wash carried out in less than the ionic strength of 5×SSC, 0.05 M sodium phosphate, 42% formamide, 0.1% SDS at a temperature of at least 38° C. and a washing step of at least 38° C. in 2×SSC

The length for a hybridizable polynucleotide is at least about 10 nucleotides. Preferably the length for a hybridizable polynucleotide is at least about 15 nucleotides; more preferably at least about 20 nucleotides; and even more preferably the length is at least 30 nucleotides.

In a sixth aspect, the present invention provides an isolated polynucleotide which encodes an EcR polypeptide of a N. viridula ecdysone receptor, wherein the polynucleotide has a sequence that hybridises under high stringency conditions to the nucleotide sequence set forth in SEQ ID No:1 or SEQ ID No:2; or a sequence fully complementary thereto, wherein high stringency conditions are a hybridisation and/or wash carried out in less than the ionic strength of 5×SSC, 0.05 M sodium phosphate, 42% formamide, 0.1% SDS at a temperature of at least 38° C. and a washing step of at least 38° C. in 2×SSC.

In a seventh aspect, the present invention provides an isolated polynucleotide which encodes a USP polypeptide of a N. viridula ecdysone receptor, wherein the polynucleotide has a sequence that hybridises under high stringency conditions to the nucleotide sequence set forth in SEQ ID No:3; or to a sequence fully complementary thereto, wherein high stringency conditions are a hybridisation and/or wash carried out in less than the ionic strength of 5×SSC, 0.05 M sodium phosphate, 42% formamide, 0.1% SDS at a temperature of at least 38° C. and a washing step of at least 38° C. in 2×SSC. In an eighth aspect the present invention provides a genetic construct comprising the isolated polynucleotide according to any previous aspect of the invention, operably linked to a promoter sequence.

In a ninth aspect, the present invention provides a recombinant cell comprising the isolated polynucleotide according to any one of the first to seventh aspects of the invention or the genetic construct according to the eighth aspect of the invention.

In a tenth aspect of the invention, there is provided an animal (such as a mammal or insect), microorganism, plant or aquatic organism, containing one or more cells according to the ninth aspect of the invention.

In an eleventh aspect, the present invention provides an isolated EcR polypeptide of the N. viridula ecdysone receptor comprising the amino acid sequence set forth in SEQ ID No: 4 or SEQ ID No:5 or a sequence at least 60% identical, preferably at least 80% identical, more preferably at least 90%, 95%, 96%, 97%, 98% or 99% identical to the sequence set forth in SEQ ID No:4 or SEQ ID No:5.

In a twelfth aspect, the present invention provides an isolated USP polypeptide of the N. viridula ecdysone receptor comprising an amino acid sequence set forth in SEQ ID No:6 or a sequence at least 60% identical, preferably at least 80% identical, more preferably at least 90%, 95%, 96%, 97%, 98% or 99% identical to the sequence set forth in SEQ ID No:6.

In a thirteenth aspect, the present invention also provides isolated fragments of the N. viridula EcR and USP polypeptides, particularly isolated fragments comprising functional domain regions.

In a fourteenth aspect, the present invention provides for the use of a polypeptide according to the eleventh or twelfth aspect of the invention in gene switching.

In a fifteenth aspect, the present invention provides a method of identifying a modulator of a N. viridula ecdysone receptor comprising:

(a) assaying the binding of a reporter ligand to a N. viridula ecdysone receptor polypeptide according to the eleventh or twelfth aspects of the present invention in the presence of a potential modulator; and

(b) assaying the binding of a reporter ligand to the ecdysone receptor polypeptide according to the eleventh or twelfth aspects of the present invention without said potential modulator; and

(c) comparing the binding of the reporter ligand in the presence of the potential modulator to the binding of the reporter ligand in the absence of the potential modulator,

wherein a difference in the level of binding indicates that said potential modulator is a modulator of N. viridula ecdysone receptor.

In a sixteenth aspect, the present invention provides a method for screening a candidate compound for its ability to interact with a N. viridula ecdysone receptor or ligand binding domain (LBD) thereof in a competitive inhibition format, the method comprising the steps of: (a) incubating with a N. viridula ecdysone receptor or LBD thereof, a candidate compound and a fluorescent compound; and (b) measuring the level of binding of the fluorescent compound to the ecdysone receptor or LBD thereof.

In a seventeenth aspect, the present invention provides a method of identifying a candidate insecticidally-active agent of a N. viridula ecdysone receptor, comprising the steps of

a) expressing the USP polypeptide in combination with an isolated N. viridula EcR polypeptide according to the eleventh aspect so as to form a complex;

b) purifying or precipitating the complex;

c) determining the three-dimensional structure of the ligand binding domain of the complex; and

d) identifying compounds which bind to or associate with the three-dimensional structure of the ligand binding domain, wherein said compounds represent candidate insecticidally-active agents.

### BRIEF DESCRIPTION OF THE FIGURES

FIGS. 1A and 1B show predicted translation initiation start sites of NvEcR10 and NvEcR11 (FIG. 1A) and NvUSP (FIG. 1B). The ATG codons in larger font denote the respective predicted translation initiation sites (TIS). Individual TIS are labelled above as NvEcR10, 11 or NvUSP. Underlined residues denote those which conform to the Drosophila consensus sequence (Liu and Xue, 2005). Underlined TGA codons in larger font highlight an upstream stop codon. (a) The italicised, smaller cased text indicates sequence which is 5′UT of the NvEcR11 cDNA. The nucleotide and amino acid sequences of FIG. 1A correspond to SEQ ID NOS:19 and 20 and those in FIG. 1B correspond to SEQ ID NOS:21 and 22.

FIG. 2 is a comparison of PCR products from genomic DNA isolated from individual insects (lanes 1-10) and from cDNA of NvEcR10 (lane A) and NvEcR11 (lane B). M1 and M2 denote the 1 kb and 100 bp ladders respectively (New England Biolabs). N denotes the negative control.

FIGS. 3A and 3B show the sequences and locations of donor and acceptor sites which yield the two different NvEcR isoforms. Italicised sequence indicates intron sequence, remaining sequence is exon sequence. Underlined text is that which was recognised by splice site predictor programs as consensus donor (a) and acceptor (b) sequences. The nucleotide and amino acid sequences of FIG. 3A correspond to SEQ ID NOS: 23 and 24 and those in FIG. 3B correspond to SEQ ID NOS: 25 and 26.

FIG. 4 depicts Coomassie blue stained SDS PAGE analysis of the purified ligand binding regions of recombinant proteins NvEcR10_{DEF}_{^{−}} NvUSP_{DEF} and NvEcR11_{DEF}_{^{−}} NvUSP_{DEF}. The proteins were purified using immobilised metal affinity column chromatography via the (His)₆ tag fused to the N terminus of the DEF domains of the EcR subunits from Myzus persicae. Mp denotes the similarly immunoaffinity tagged, ligand binding regions of Myzus persicae. 11 denotes NvEcR11_{DEF}_{^{−}}NvUSP_{DEF} and 10 denotes NvEcR10_{DEF}_{^{−}}NvUSP_{DEF}. The DEF domains of the two NvEcR isoforms and for NvUSP are defined by tables 1 and 2 taken together with the NvEcR10 polynucleotide sequence of SEQ ID No:1, the NvEcR10 amino acid sequence of SEQ ID No:4, the NvEcR11 polynucleotide sequence of SEQ ID No:2 and the NvEcR11 amino acid sequence of SEQ ID No:5, the NvUSP polynucleotide sequence of SEQ ID No: 3 and the NvUSP amino acid sequence of SEQ ID No: 6.

FIG. 5 shows [³H] Ponasterone A binding data used in Kd determination for the purified recombinant heterodimers NvEcR10_{DEF}_{^{−}}NvUSP_{DEF} and NvEcR11_{DEF}_{^{−}}NvUSP_{DEF} (Kd curves). Rt=concentration of active receptor. L=free [³H] Ponasterone A. All values in tables are given in pM.

FIGS. 6A and 6B depict the ability of muristerone A, ponasterone A, 20-hydroxyecdysone, inokosterone, and α-ecdysone (from concentrated ethanolic stock solutions) to compete with 1.3 nM [³H] Ponasterone A binding to the purified recombinant heterodimers NvEcR10_{DEF}_{^{−}}NvUSP_{DEF} and NvEcR11_{DEF}_{^{−}}NvUSP_{DEF}, respectively. Each inhibitor competes with 1.3 nM [³H] ponasterone A binding. Binding is expressed as a percentage of that observed in control incubations containing no competitor but the appropriate concentration of ethanol.

### DETAILED DESCRIPTION OF THE INVENTION

The present invention advantageously provides isolated polynucleotide and polypeptide sequences of two novel EcR isoforms and a novel ultraspiracle (USP) from N. viridula. The polynucleotides and polypeptides of the present invention are useful in screening methods, particularly in identifying modulating agents of such receptors.

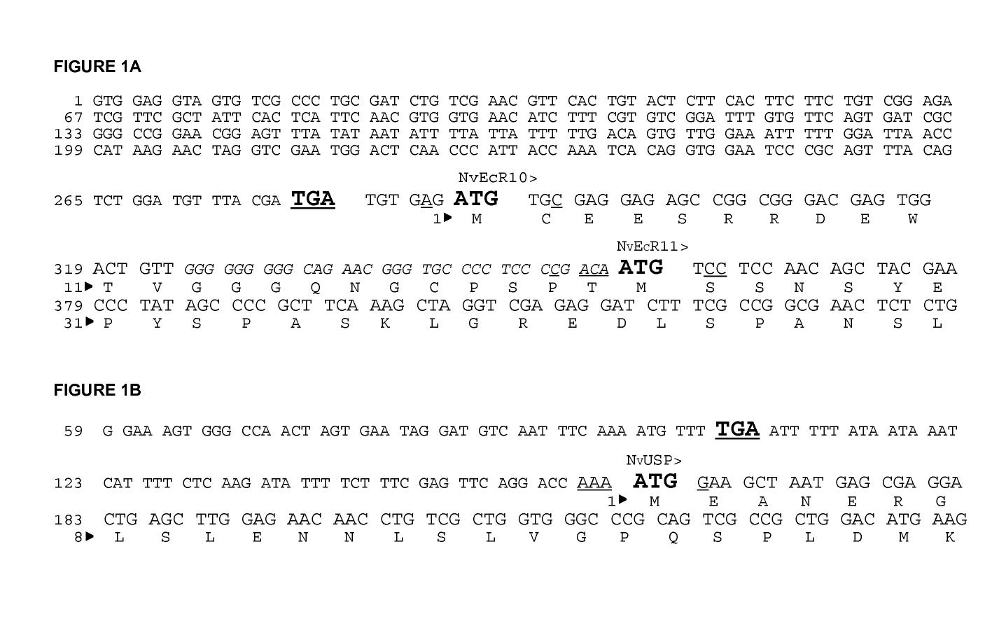

The first novel EcR isoform NvEcR10, has the polynucleotide sequence of SEQ ID No:1 and the amino acid sequence of SEQ ID No:4. The second novel EcR isoform NvEcR11 has the polynucleotide sequence of SEQ ID No:2 and the amino acid sequence of SEQ ID No:5. The difference between NvEcR10 and NvEcR11 at their N-termini is due to the utilisation of two different translational initiation sites (FIG. 1). The EcR isoforms also differ in that NvEcR10 contains two additional amino acid residues (ValSer) in Domain E, the ligand binding domain, (at positions 237 and 238 of SEQ ID No:4). This difference in amino acid residues between NvEcR10 and NvEcR11 which resides in the E domain is a region of the molecule that is typically not affected by differential splicing. In fact, the only other known possible occurrence of differential splicing in the E domain of an EcR has been in that of Bombyx mori (Swevers et al., 1995), which has not been characterised or confirmed. In the case of N. viridula however, differential splicing in the E domain has been strongly supported as being responsible for generating the two isoforms NvEcR10 and NvEcR11. Furthermore, the ligand binding domains of these isoforms have undergone preliminary characterisation. In view of the finding of these different ligand binding domains of N. viridula EcR, it would be preferable to identify modulators, such as an insecticidally-active agent of N. viridula ecdysone receptor that target both isoforms of the N. viridula EcR protein.

One aspect of the present invention provides an isolated polynucleotide comprising a nucleotide sequence which encodes or is complementary to a sequence which encodes an EcR polypeptide of an N. viridula ecdysone receptor, wherein the polynucleotide comprises a nucleic acid sequence at least 60%, preferably at least 80% identical, more preferably at least 90%, 95%, 96%, 97%, 98% or 99% identical to the sequence set forth in SEQ ID No:1 or SEQ ID No:2 and most preferably, the sequence set forth in SEQ ID No:1 or SEQ ID No:2.

In a second aspect, the present invention provides an isolated polynucleotide comprising a nucleotide sequence which encodes or is complementary to a sequence which encodes a USP polypeptide of a N. viridula ecdysone receptor, wherein the polynucleotide comprises a nucleic acid sequence at least 60%, preferably at least 80% identical, more preferably at least 90%, 95%, 96%, 97%, 98% or 99% identical to the sequence set forth in SEQ ID No:3, and most preferably, the sequence set forth in SEQ ID No:3.

Those skilled in the art of the present invention will be aware that variants of the nucleotide sequences set forth in any one of SEQ ID No:1 to SEQ ID No: 3 or fragments thereof may be isolated by hybridization under suitable stringency conditions as exemplified herein. Such variants include any genomic sequences, cDNA sequences, mRNA or other isolated nucleic acid molecules derived from the polynucleotides exemplified herein by the Sequence Listing. Additional variants are not excluded.

Accordingly, the isolated polynucleotides of the invention may comprise a fragment of a nucleotide sequence encoding a full-length novel EcR isoform or a novel ultraspiracle (USP) from N. viridula. It is to be understood that a “fragment” of a nucleotide sequence encoding a N. viridula EcR polypeptide or a USP polypeptide of a N. viridula ecdysone receptor refers to a nucleotide sequence encoding a part or fragment of such a receptor which is capable of binding or associating with an insect steroid or an analogue thereof, or a modulator of the receptor, such as a candidate insecticidally active compound. Fragments of a nucleotide sequence would generally comprise in excess of twenty contiguous nucleotides derived from the base sequence and may encode one or more domains of a N. viridula EcR or a USP polypeptide of a N. viridula ecdysone receptor.

In a third aspect, the present invention provides an isolated polynucleotide comprising a nucleotide sequence which encodes or is complementary to a sequence which encodes an EcR polypeptide of an N. viridula ecdysone receptor, the polypeptide comprising an amino acid sequence at least 60%, preferably at least 80% identical, more preferably at least 90%, 95%, 96%, 97%, 98% or 99% identical to the sequence set forth in SEQ ID No:4 or SEQ ID No:5 and most preferably, the amino acid sequence set forth in SEQ ID No:4 or SEQ ID No:5.

In a fourth aspect, the present invention provides an isolated polynucleotide comprising a nucleotide sequence which encodes or is complementary to a sequence which encodes a USP polypeptide of a N. viridula ecdysone receptor, the polypeptide comprising an amino acid sequence at least 60%, preferably at least 80% identical, more preferably at least 90%, 95%, 96%, 97%, 98% or 99% identical to the sequence set forth in SEQ ID No:6 and most preferably, the amino acid sequence set forth in SEQ ID No:6.

The first novel EcR isoform NvEcR10 has the amino acid sequence of SEQ ID No:4. The second novel EcR isoform NvEcR11 has the amino acid sequence of SEQ ID No:5. The USP polypeptide of a N. viridula ecdysone receptor has the amino acid sequence of SEQ ID No:6. Therefore, two ecdysone receptor heterodimers, NvEcR10-NvUSP and NvEcR11-NvUSP can form from these polypeptides.

In determining whether or not two polynucleotide sequences fall within certain percentage limits, those skilled in the art will be aware that it is necessary to conduct a side-by-side comparison or multiple alignment of sequences. In such comparisons or alignments, differences may arise in the positioning of non-identical residues, depending upon the algorithm used to perform the alignment. In the present context, reference to a percentage identity between two or more polynucleotide sequences shall be taken to refer to the number of identical residues between said sequences as determined using any standard algorithm known to those skilled in the art. For example, nucleotide sequences may be aligned and their identity calculated using the BESTFIT programme or other appropriate programme of the Computer Genetics Group, Inc., University Research Park, Madison, Wis., United States of America (Devereux et al., 1984).

In a fifth aspect, the present invention provides an isolated polynucleotide comprising a sequence that is at least 10 nucleotides in length capable of hybridising under at least high stringency conditions to the nucleotide sequence set forth in SEQ ID No:1, SEQ ID No:2 or SEQ ID No:3; or to a complementary nucleotide sequence thereto, wherein high stringency conditions are a hybridisation and/or wash carried out in less than the ionic strength of 5×SSC, 0.05 M sodium phosphate, 42% formamide, 0.1% SDS at a temperature of at least 38° C. and a washing step of at least 38° C. in 2×SSC

The length for a hybridizable polynucleotide is at least about 10 nucleotides. Preferably the length for a hybridizable polynucleotide is at least about 15 nucleotides; more preferably at least about 20 nucleotides; and even more preferably the length is at least 30 nucleotides.

In a sixth aspect, the present invention provides an isolated polynucleotide which encodes an EcR polypeptide of a N. viridula ecdysone receptor, wherein the polynucleotide has a sequence that hybridises under high stringency conditions to the nucleotide sequence set forth in SEQ ID No:1 or SEQ ID No:2; or a sequence fully complementary thereto, wherein high stringency conditions are a hybridisation and/or wash carried out in less than the ionic strength of 5×SSC, 0.05 M sodium phosphate, 42% formamide, 0.1% SDS at a temperature of at least 38° C. and a washing step of at least 38° C. in 2×SSC.

In a seventh aspect, the present invention provides an isolated polynucleotide which encodes a USP polypeptide of a N. viridula ecdysone receptor, wherein the polynucleotide has a sequence that hybridises under high stringency conditions to the nucleotide sequence set forth in SEQ ID No:3; or to a sequence fully complementary thereto, wherein high stringency conditions are a hybridisation and/or wash carried out in less than the ionic strength of 5×SSC, 0.05 M sodium phosphate, 42% formamide, 0.1% SDS at a temperature of at least 38° C. and a washing step of at least 38° C. in 2×SSC.

A polynucleotide is “hybridizable” to another polynucleotide, such as a cDNA, genomic DNA, or RNA, when a single stranded form of the nucleic acid molecule can anneal to the other nucleic acid molecule under the appropriate conditions of temperature and solution ionic strength (see Sambrook et al., 1989). Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein (entirely incorporated herein by reference). The conditions of temperature and ionic strength determine the “stringency” of the hybridization.

Stringency conditions can be adjusted to screen for moderately similar fragments, such as homologous sequences from distantly related organisms, to highly similar fragments, such as genes that duplicate functional enzymes from closely related organisms. For preliminary screening for homologous nucleic acids, low stringency hybridization conditions, corresponding to a hybridisation temperature of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tm, e.g., 40% formamide, with 5× or 6×SCC. High stringency hybridization conditions correspond to the highest Tm, e.g., 50% formamide, 5× or 6×SSC.

Hybridization requires that the two polynucleotides contain complementary sequences, although depending on the stringency of the hybridization, mismatches between bases are possible. The term “complementary” is used to describe the relationship between nucleotide bases that are capable of hybridizing to one another. For example, with respect to DNA, adenosine is complementary to thymine and cytosine is complementary to guanine. Accordingly, the instant invention also includes isolated nucleic acid fragments that are complementary to the complete sequences as disclosed or used herein as well as those substantially similar nucleic acid sequences.

In a specific embodiment of the fifth, sixth or seventh aspects of the invention, polynucleotides are detected by employing hybridization conditions comprising a hybridization step at of 38° C., and utilizing conditions as set forth above. In a preferred embodiment, the hybridization temperature is 40° C.; in a more preferred embodiment, the hybridization temperature is 42° C.; in an even more preferred embodiment, the hybridization temperature is 44° C.

Post-hybridization washes also determine stringency conditions. One set of preferred conditions uses a series of washes starting with 2×SSC, 0.1% SDS at room temperature for 15 minutes (min), then repeated with 2×SSC, 0.1% SDS at 38° C. for 30 minutes.

The appropriate stringency for hybridizing polynucleotides depends on the length of the nucleic acids and the degree of complementation, variables well known in the art. The greater the degree of similarity or homology between two nucleotide sequences, the greater the value of the Tm for hybrids of nucleic acids having those sequences. The relative stability (corresponding to higher Tm) of nucleic acid hybridizations decreases in the following order: RNA: RNA, DNA: RNA, DNA: DNA. For hybrids of greater than 100 nucleotides in length, equations for calculating Tm have been derived (see Sambrook et al., supra, 9.50-9.51). For hybridization with shorter nucleic acids, i.e., oligonucleotides, the position of mismatches becomes more important, and the length of the oligonucleotide becomes an important factor in determining specificity (see Sambrook et al., supra, 11.7-11.8). As other factors may significantly affect the stringency of hybridization, including base composition and length of the complementary strands, presence of organic solvents and extent of base mismatching, the combination of parameters is more important than the absolute measure of any one.

Furthermore, the skilled artisan will recognize that the temperature and wash solution salt concentration may be adjusted as necessary according to factors such as length of the probe.

The polynucleotides of the present invention are also useful for developing genetic constructs which comprise and preferably express the novel ecdysone receptor isoforms or the novel ultraspiracle (USP) from N. viridula, thereby providing for the production of the recombinant polypeptides in isolated cells or transformed tissues.

Accordingly, in an eighth aspect the present invention provides a genetic construct comprising the isolated polynucleotide according to any previous aspect of the invention, operably linked to a promoter sequence.

Preferably, the polynucleotide is in an expressible format, such that it is possible to produce a recombinant polypeptide therefrom. Reference herein to a “promoter” is to be taken in its broadest context and includes the transcriptional regulatory sequences of a classical genomic gene, including the TATA box which is required for accurate transcription initiation in a eukaryotic cell, with or without a CCAAT box sequence. Promoters may be cell, tissue, organ or system specific, or may be non-specific. Using specific promoters, the expression of a bioactive agent or other polypeptide encoded by a structural gene to which the promoter is operably connected may be targeted to a desired cellular type.

In the present context, the term “promoter” is also used to describe a synthetic or fusion molecule, or derivative which confers, activates or enhances expression in a cell in response to an external stimulus. Accordingly, the promoter may include further regulatory elements (i.e. upstream activating sequences, enhancers and silencers) which alter gene expression in response to developmental and/or external stimuli, or in a tissue-specific manner. Preferred promoters may contain copies of one or more specific regulatory elements, in particular steroid responsive elements (SREs) or hormone-responsive elements (HREs), to further enhance expression and/or to alter the spatial expression and/or temporal expression pattern.

Placing an isolated polynucleotide of the present invention operably under the control of a promoter sequence means positioning said gene or isolated polynucleotide such that its expression is controlled by the promoter sequence. Promoters are generally positioned 5′ (upstream) to the genes that they control. In the construction of heterologous promoter/structural gene combinations it is generally preferred to position the promoter at a distance from the gene transcription start site that is approximately the same as the distance between that promoter and the gene it controls in its natural setting, i.e., the gene from which the promoter is derived. As is known in the art, some variation in this distance can be accommodated without loss of promoter function. Similarly, the preferred positioning of a regulatory sequence element with respect to a heterologous gene to be placed under its control is defined by the positioning of the element in its natural setting, i.e., the genes from which it is derived. Again, as is known in the art, some variation in this distance can also occur.

Those skilled in the art will recognise that the choice of promoter will depend upon the nature of the cell being transformed and when expression is required. Furthermore, it is well-known in the art that the promoter sequence used in the expression vector will also vary depending upon the level of expression required and whether expression is intended to be constitutive or regulated.

For expression in eukaryotic cells, the genetic construct generally comprises, in addition to the polynucleotide of the invention, a promoter and optionally other regulatory sequences designed to facilitate expression of said polynucleotide. The promoter may be derived from a genomic clone which normally encodes the expressed protein or alliteratively, it may be a heterologous promoter derived from another genetic source. Promoter sequences suitable for expression of genes in eukaryotic cells are well-known in the art.

Suitable promoters for use in eukaryotic expression vectors include those capable of regulating expression in mammalian cells, insect cells such as Sf9, Sf21. (Spodoptera frugiperda) or Hi-5, (Trichoplusia ni) yeast cells and plant cells. Preferred promoters for expression in eukaryotic cells include the p10 promoter, MMTV promoter, polyhedron promoter, the SV40 early promoter and the cytomegalovirus (CMV-IE) promoter, promoters derived from immunoglobulin-producing cells (see, U.S. Pat. No. 4,663,281), polyoma virus promoters, and the LTR from various retroviruses (such as murine leukemia virus, murine or Rous sarcoma virus and HIV), amongst others (See, Enhancers and Eukaryotic Gene Expression, Cold Spring Harbor Press, New York, 1983, which is incorporated herein by reference). Examples of other expression control sequences are enhancers or promoters derived from viruses, such as SV40, Adenovirus, Bovine Papilloma Virus, and the like.

Wherein the expression vector is intended for the production of recombinant protein, the promoter is further selected such that it is capable of regulating expression in a cell which is capable of performing any post-translational modification to the polypeptide which may be required for the subject recombinant polypeptide to be functional, such as N-linked glycosylation. Cells suitable for such purposes may be readily determined by those skilled in the art. By way of exemplification, baculovirus may be used to express recombinant polypeptides using standard protocols in Sf9, Sf21 or Hi-5 insect cells.

Numerous expression vectors suitable for the present purpose have been described and are readily available. The expression vector may be based upon pFastBacDual, pCR-TOPO2.1, piRES, and pDual.

Examples of eukaryotic cells contemplated herein to be suitable for expression include mammalian, yeast, insect, plant cells or cell lines such as COS, VERO, HeLa, mouse C127, Chinese hamster ovary (CHO), WI-38, baby hamster kidney (BHK), MDCK, 3T3, HEK, Sf21 (insect) Sf9 (insect) or Hi-5 (insect) cell lines. Such cell lines are readily available to those skilled in the art.

The prerequisite for expression in prokaryotic cells such as Escherichia coli is the use of a strong promoter with an effective ribosome binding site. Typical promoters suitable for expression in bacterial cells such as E. coli include, but are not limited to, the lacZ promoter, temperature-sensitive A, or AR promoters, T7 promoter or the IPTG-inducible tac promoter. A number of vector systems for expressing the nucleic acid molecule of the invention in E. coli are well-known in the art and include pETDuet-1 (Novagen), pRSETh (Invitrogen), pET-based vectors or pGEX-4T, and pACYC are others described for example in Ausubel et al. (1992).

Suitable prokaryotic cells include strains of Corynebacterium, Salmonella, Escherichia coli, for example, but not limited to, BL21 (DE3), Bacillus sp. and Pseudomonas sp, amongst others. Bacterial strains which are suitable for the present purpose are well-known in the relevant art (Ausubel et al., 1992).

The genetic constructs described herein may further comprise genetic sequences corresponding to a bacterial origin of replication and/or a selectable marker gene such as an antibiotic-resistance gene, suitable for the maintenance and replication of said genetic construct in a prokaryotic or eukaryotic cell, tissue or organism. Such sequences are well-known in the art.

Selectable marker genes include genes which when expressed are capable of conferring resistance on a cell to a compound which would, absent expression of said selectable marker gene, prevent or slow cell proliferation or result in cell death. Preferred selectable marker genes contemplated herein include, but are not limited to antibiotic-resistance genes such as those conferring resistance to ampicillin, Claforan, gentamycin, G-418, hygromycin, rifampicin, kanamycin, neomycin, spectinomycin, tetracycline or a derivative or related compound thereof or any other compound which may be toxic to a cell. The origin of replication or a selectable marker gene will be spatially-separated from those genetic sequences which encode the recombinant receptor polypeptide or fusion polypeptide comprising same.

Preferably, the genetic constructs of the invention, including any expression vectors, are capable of introduction into, and expression in, an in vitro cell culture, or for introduction into, with or without integration into the genome of a cultured cell, cell line or transgenic animal.

In a particularly preferred embodiment, the expression vector is selected from the group consisting of pFastBacDual, piRES, pDual, pETDuet-1 (Novagen), pRSETh (Invitrogen), pET-based vectors or pGEX-4T, and pACYC

In a ninth aspect, the present invention provides a recombinant cell comprising the isolated polynucleotide according to any one of the first to seventh aspects of the invention or the genetic construct according to the eighth aspect of the invention.

As used herein, the term “recombinant cell” shall be taken to refer to a single cell, or a cell lysate, or a tissue, organ or whole organism comprising same, including a tissue, organ or whole organism comprising a clonal group of cells or a heterogenous mixture of cell types, which may be a prokaryotic or eukaryotic cell that comprises a polynucleotide of the present invention or a fragment thereof that has been incorporated artificially into the cell, preferably the polynucleotide is incorporated in the cell with a suitable vector so that it can replicate and express itself many times.

As used herein, the term “recombinant cell” is meant to also include the progeny of a transformed cell.

In a preferred embodiment, the recombinant cell of the present invention expresses the polypeptide encoded by the polynucleotides of the present invention.

In a preferred embodiment, the cell expresses an EcR isoform or the novel ultraspiracle (USP) from Nezara viridula, or a fragment thereof and comprises a nucleic acid sequence encoding a bioactive molecule or a reporter molecule,

To produce the recombinant cells of the invention, host cells are transfected or co-transfected or transformed with nucleotide sequences containing the DNA segments of interest (for example, EcR isoforms or the novel ultraspiracle (USP) from Nezara viridula,) by well-known methods, which vary depending on the type of cellular host. For example, calcium chloride transfection is commonly utilized for prokaryotic cells, whereas lipofection, CeltFectin or calcium phosphate treatment, are often used for other cellular hosts. See, generally, Sambrook et al., (1989); Ausubel et al., (1992); and Potrykus (1990). Other transformation techniques include electroporation, DEAE-dextran, microprojectile bombardment, lipofection, microinjection, and others.

In a tenth aspect of the invention, there is provided an animal (such as a mammal or insect), microorganism, plant or aquatic organism, containing one or more cells according to the ninth aspect of the invention.

Reference to plants, microorganisms and aquatic organisms includes any such organisms. Preferably, the plant is selected from the group consisting of rice, cotton, beans, cabbage, potatoes, mango, tomatoes, citrus fruits and nuts.

The present invention further clearly provides for the isolation of EcR and USP polypeptides of the N. viridula ecdysone receptor.

Accordingly, in an eleventh aspect, the present invention provides an isolated EcR polypeptide of the N. viridula ecdysone receptor comprising the amino acid sequence set forth in SEQ ID No: 4 or SEQ ID No:5 or a sequence at least 60% identical, preferably at least 80% identical, more preferably at least 90%, 95%, 96%, 97%, 98% or 99% identical to the sequence set forth in SEQ ID No:4 or SEQ ID No:5.

In a twelfth aspect, the present invention provides an isolated USP polypeptide of the N. viridula ecdysone receptor comprising an amino acid sequence set forth in SEQ ID No:6 or a sequence at least 60% identical, preferably at least 80% identical, more preferably at least 90%, 95%, 96%, 97%, 98% or 99% identical to the sequence set forth in SEQ ID No:6.

In determining whether or not two amino acid sequences fall within certain percentage limits, those skilled in the art will be aware that it is necessary to conduct a side-by-side comparison or multiple alignment of sequences. In such comparisons or alignments, differences will arise in the positioning of non-identical residues, depending upon the algorithm used to perform the alignment. In the present context, reference to a percentage identity or similarity between two or more amino acid sequences shall be taken to refer to the number of identical and similar residues respectively, between said sequences as determined using any standard algorithm known to those skilled in the art. For example, amino acid sequence identities or similarities may be calculated using the GAP programme and/or aligned using the PILEUP programme of the Computer Genetics Group, Inc., University Research Park, Madison, Wis., United States of America (Devereux et al., 1984). The GAP programme utilizes the algorithm of Needleman and Wunsch (1970) to maximise the number of identical/similar residues and to minimise the number and length of sequence gaps in the alignment. Alternatively or in addition, wherein more than two amino acid sequences are being compared, the Clustal W programme of Thompson et al., (1994) is used.

Insect steroid receptors, such as the N. viridula EcR and USP polypeptide subunits of the N. viridula ecdysone receptor, are characterized by functional ligand-binding domains, and DNA-binding domains. The N. viridula ecdysone receptor contains a DNA-binding domain (Domain C), and a ligand-binding domain (Domain E), separated and flanked by additional domains (see Tables 1 and 2). The C domain preferably comprises a zinc-finger DNA-binding domain which is usually hydrophilic, having high cystine, lysine and arginine content. The E domain is a globular domain that binds ligands, coactivator proteins and corepressor proteins. Amino acid residues proximal to the C domain comprise a region initially defined as separate A and B domains. Region D separates the more conserved domains C and E. Region D typically has a hydrophilic region whose predicted secondary structure is rich in turns and coils.

In a thirteenth aspect, the present invention also provides isolated fragments of the N. viridula EcR and USP polypeptides, particularly isolated fragments comprising functional domain regions.

Any of the functional domains may be used to construct chimeric nuclear receptors by functionally linking them to components of other nuclear receptors for use, e.g. in ecdysone switches. Fragments of the N. viridula EcRs and USP e.g. encompassing the D and E or just the E domains may be expressed to produce functional ligand binding protein for use in binding assays and screens for ligands exhibiting new chemistries.

Preferred fragments of the polypeptides of the present invention include one or more regions or domains which are involved in the interaction or association between the monomeric polypeptide subunits of a multimeric receptor and/or which are involved in the interaction or association between (i) a cognate steroid or receptor ligand or cis-acting DNA sequence; and (ii) said monomeric polypeptide subunits or the receptor per se. In a particularly preferred embodiment, the fragments comprise the DNA-binding domain, linker domain or a part thereof, or ligand-binding domain (eg. hormone-binding domain) of a EcR polypeptide or novel EcR from N. viridula receptor polypeptide. Preferably, the polypeptide retains the biological activity of the novel ecdysone receptor isoforms and a novel ultraspiracle (USP) from N. viridula, it is then required to include at least a ligand-binding region comprising the ligand-binding domain and at least a part of the linker domain of the EcR polypeptide subunit, optionally in association with a ligand-binding region comprising at least the ligand-binding domain and at least a part of the linker domain of the EcR partner protein (USP polypeptide) subunit of said receptor. Additional fragments are not excluded.

Accordingly, the present invention also provides an isolated EcR-USP heterodimer comprising an EcR polypeptide of the N. viridula ecdysone receptor set forth in SEQ ID No:4 or SEQ ID No:5 or ligand binding domain thereof in association with an EcR partner protein (USP polypeptide) set forth in SEQ ID No:6 or ligand binding domain thereof.

As is known in the field ecdysone receptors and their functional domains are employed as components of ecdysone switches for the control of therapeutic genes in mammalian cells (Lafont & Dinan, 2003; Yang et al., 1986) and for control of transgenes more generally in agriculturally important species, both animal and plant (Lafont & Dinan, 2003; Padidam et al., 2003).

In a fourteenth aspect, the present invention provides for the use of a polypeptide according to the eleventh or twelfth aspect of the invention in gene switching.

Preferably, the recombinant or isolated N. viridula ecdysone receptor polypeptide as described herein, is thermostable. By “thermostable” is meant that polypeptide does not exhibit reduced activity at bacterial, plant or animal physiological temperatures above about 28° C. or above about 30° C. The thermostability of insect steroid hormone receptors also refers to the capacity of such receptors to bind to ligand-binding domains or regions or to transactivate genes linked to insect steroid hormone response elements at bacterial, plant or animal physiological temperatures above about 28° C. or above about 30° C.

The present invention clearly extends to variants of the polypeptides of the present invention. The polypeptide may be substantially free of naturally associated with insect cell components, or may be in combination with a partner protein which associates with the insect steroid receptor so as to confer enhanced affinity for insect steroid response elements, enhanced affinity for insect steroids or analogues thereof. For example, the amino acid sequences (eg SEQ ID No:s 4 to 6) described herein may be varied by the deletion, substitution or insertion of one or more amino acids.

In one embodiment, amino acids of the polypeptides described herein may be replaced by other amino acids having similar properties, for example hydrophobicity, hydrophilicity, hydrophobic moment, charge or antigenicity, and so on.

Substitutions encompass amino acid alterations in which an amino acid of the base polypeptide is replaced with a different naturally-occurring or a non-conventional amino acid residue. Such substitutions may be classified as “conservative”, in which case an amino acid residue contained in the base polypeptide is replaced with another naturally-occurring amino acid of similar character, for example Gly-Ala, Lys-Arg or Phe-Trp-Tyr.

Substitutions encompassed by the present invention may also be “non-conservative”, in which an amino acid residue which is present in the base polypeptide is substituted with an amino acid having different properties, such as a naturally-occurring amino acid from a different group (eg. substituted a charged or hydrophobic amino acid with alanine), or alternatively, in which a naturally-occurring amino acid is substituted with a non-conventional amino acid.

Those skilled in the art will be aware that several means are available for producing variants of the described polypeptide sequences, when provided with the nucleotide sequence of the polynucleotide which encodes said polypeptide, for example site-directed mutagenesis of DNA and polymerase chain reaction utilising mutagenised oligonucleotide primers, amongst others.

Such polypeptide variants which are capable of binding insect steroids or receptor modulators and ligands clearly form part of the present invention. Assays to determine such binding may be carried out according to procedures well known in the art.

The polypeptides of the present invention can include fusion polypeptides, such as but not limited to, fusion between different regions of different insect receptor polypeptides. Different domains of the polypeptides of the invention may be replaced with foreign polypeptide fragments. Fusion polypeptides of the present invention can include fusion with a second polypeptide, for example a detectable reporter polypeptide such as—β-galactosidase, β-glucuronidase, luciferase or other enzyme, or a FLAG peptide, hapten peptide such as a poly-lysine or poly-histidine or other polypeptide molecule.

In order to produce a fusion polypeptide, the nucleic acid molecule which encodes the polypeptide of the invention, or an analogue or derivative thereof, is cloned adjacent to a second nucleic acid molecule encoding the second polypeptide, optionally separated by a spacer nucleic acid molecule which encodes one or more amino acids (eg: poly-lysine or poly histidine, amongst others), such that the first coding region and the second coding region are in the same open reading frame, with no intervening stop codons between the two coding regions. When translated, the polypeptide thus produced comprises a fusion between the polypeptide products of the first and second coding regions. Wherein a spacer nucleic acid molecule is utilised in the genetic construct, it may be desirable for said spacer to at least encode an amino acid sequence which is cleavable to assist in separation of the fused polypeptide products of the first and second coding regions, for example a thrombin cleavage site.

A genetic construct which encodes a fusion polypeptide further comprises at least one start codon and one stop codon, capable of being recognised by the cell's translational machinery in which expression is intended.

Preferably, a genetic construct which encodes a fusion polypeptide may be further modified to include a genetic sequence which encodes a targeting signal placed in-frame with the coding region of the nucleotide sequence encoding the fusion polypeptide, to target the expressed recombinant polypeptide to the extracellular matrix or other cell compartment. More preferably, the genetic sequence encoding a targeting signal is placed in-frame at the 5′-terminus or the 3′-terminus, but most preferably at the 5′-terminus, of the coding region of the nucleotide sequence which encodes the fusion polypeptide.

Methods for the production of a fusion polypeptide are well-known to those skilled in the art.

In a further embodiment, the biological function of the polypeptides as herein described can be modulated by making specific changes (e.g. addition, substitution or deletion) to only those amino-acids within each domain that are critical for determining the relevant function (eg. ligand-binding activity, DNA binding site affinity, etc), such as by site-directed mutagenesis. Accordingly, the polypeptides of the present invention can differ in amino acid sequence and/or exhibit different biological properties to a naturally-occurring N. viridula ecdysone receptor.

As with other aspects of the invention, the variants described herein may be produced as recombinant polypeptides or in transgenic organisms, once the defined recombinant polynucleotides are introduced into a suitable host cell and expressed therein.

The N. viridula ecdysone receptor polypeptides described herein may be purified by standard techniques, such as column chromatography (using various matrices which interact with the protein products, such as ion exchange matrices, hydrophobic matrices and the like), affinity chromatography utilizing antibodies specific for the protein or other ligands such as dyes or insect steroids which bind to the protein. Wherein the recombinant polypeptide is expressed as a fusion polypeptide, it is also possible to purify the fusion polypeptide based upon its properties (eg size, solubility, charge etc).

Alternatively, the fusion polypeptide may be purified based upon the properties of the non-receptor moiety of said fusion polypeptide, for example substrate affinity. Once purified, the fusion polypeptide may be cleaved to release the intact polypeptide of the invention.

Alternatively, polypeptides may be synthesized by standard protein synthetic techniques as are well known in the art.

In a preferred embodiment, the isolated polypeptides of the invention are provided as a precipitate or crystallized by standard techniques, preferably for X-ray crystal structure determination.

The N. viridula ecdysone receptor polypeptides of the invention or ligand binding domains thereof, or their complexes with EcR partner proteins, such as USP polypeptide of N. viridula ecdysone receptor, or ligand binding domains thereof, which confer enhanced affinity for insect steroid response elements or partner proteins (USP polypeptides) or ligands, are particularly useful to model three-dimensional structure of the receptor ligand-binding region. In this embodiment of the invention, modulators of a N. viridula ecdysone receptor, such as but not limited to, insecticidal agents may be produced which bind to, or otherwise interact with, the ligand-binding region of the receptor, and preferably interfere with ligand binding. In the same way, agents may be developed which have a potentiated interaction with the insect steroid receptor over and above that of the physiological insect steroid which binds to the receptor.

Accordingly in a fifteenth aspect, the present invention provides a method of identifying a modulator of a N. viridula ecdysone receptor comprising:

(a) assaying the binding of a reporter ligand to a N. viridula ecdysone receptor polypeptide according to the eleventh or twelfth aspects of the present invention in the presence of a potential modulator; and

(b) assaying the binding of a reporter ligand to the ecdysone receptor polypeptide according to the eleventh or twelfth aspects of the present invention without said potential modulator; and

(c) comparing the binding of the reporter ligand in the presence of the potential modulator to the binding of the reporter ligand in the absence of the potential modulator,

wherein a difference in the level of binding indicates that said potential modulator is a modulator of N. viridula ecdysone receptor.

Preferably, the N. viridula ecdysone receptor polypeptide of the present invention comprises a complex of an EcR and a USP polypeptide. More preferably, the reporter ligand is [³H] Ponasterone A or a fluorescent conjugate, such as any one of the fluorescent conjugates as described in WO 2005/054271 titled “Assay for ligands of the Ecdysone receptors” the entire contents of which are incorporated herein.

WO 2005/054271 describes suitable fluorescent conjugates that are useful as ligands in in vitro ligand binding assays, include fluorescence polarization (FP) assays for ecdysone receptor ligands. The FP format is homogenous, i.e., the binding reaction and FP measurement of each assay is performed in the same compartment (e.g. a single well in a multiwell plate).

The assay is therefore ideally suited to the miniaturization and automation that underpins industrial high throughput screening programs. The fluorescent compounds can, for example, be prepared by reacting a reactive group in the fluorescent moiety with a nucleophilic group in the compound that binds to the N. viridula ecdysone receptor of the present invention. It is further preferred that the fluorescent moiety is selected from the group consisting of unsubstituted and substituted fluorescein moieties, unsubstituted and substituted dansyl moities, and unsubstituted and substituted coumarin moieties. The fluorescent moiety may be attached by derivatisation of a hydroxyl group on the alkyl side chain of an ecdysteroid moiety that is capable of binding to an ecdysone receptor or ligand binding domain thereof. More preferably, fluorescent moiety is attached to the ecdysteroid by derivatisation of a reactive primary hydroxyl group on C-26 such as occurs in inokosterone, 26-hydroxyecdysone, 20,26-dihydroxyecdysone, makisterone B, amarasterone A, amarasterone B, ajugasterone B, sidasterone A, sidasterone B and 26-hydroxy-polypodine B. In an alternative embodiment the fluorescent moiety is attached by derivatisation of a hydroxyl group at C-25 of an ecdysteroid selected from the group consisting of 20-hydroxyecdysone, makisterone A, polypodine B and rapisteronc D.

WO 2005/05427 also provides assays for screening compounds for their ability to interact with Ecdysone receptors. The assays described in WO 2005/054271 are also useful for screening and identifying modulating agents, including insecticidally-active compounds such as ligands which bind and either agonise or antagonise the N. viridula ecdysone receptors of the present invention.

Accordingly, in a sixteenth aspect, the present invention provides a method for screening a candidate compound for its ability to interact with a N. viridula ecdysone receptor or ligand binding domain (LBD) thereof in a competitive inhibition format, the method comprising the steps of: (a) incubating with a N. viridula ecdysone receptor or LBD thereof, a candidate compound and a fluorescent compound as described in WO 2005/05427; and (b) measuring the level of binding of the fluorescent compound to the ecdysone receptor or LBD thereof.

As used herein a “modulator” is a compound or molecule that agonises or antagonises the binding properties and/or biological activity of a N. viridula ecdysone receptor. Preferred potential modulators according to this embodiment include by way of example ecdysteroids such as 20-hydroxyecdysone, muristerone A, ponasterone A, ajugasterone C polypodine B. The reporter ligand may be any ligand that is known to bind to N. viridula ecdysone receptor, which binding may be monitored or assayed readily. Preferably, the reporter ligand is [³H] ponasterone A or fluorescent ecdysteroid conjugates such as MB4628, MB4592, MB4603 or MB4622 as described in WO 2005/054271. Standard methods can be used to assay the binding of the reporter ligand.

This embodiment of the invention may be applied directly to the identification of potential insecticidally-active compounds or alliteratively, modified for such purposes by assaying for the binding (direct or indirect) of the N. viridula ecdysone receptor polypeptide of the invention to a steroid response element (SRE). According to this alternative embodiment, the binding assayed in the presence or absence of a potential insecticidally-active compound is compared, wherein a difference in the level of binding indicates that the candidate compound possesses potential insecticidal activity.

Accordingly, substances may be screened for insecticidal activity by assessing their ability to bind, in vivo or in vitro, to the intact N. viridula ecdysone receptor or alternatively, the ligand-binding regions of the N. viridula ecdysone receptor polypeptide (eg. domain D linked to domain E or domains C, D and E of NvEcR linked with NvUSP to form a heterodimer or ligand binding domains (E) of N. viridula EcR and N. viridula USP.

An example of this embodiment may, for instance, involve binding the N. viridula ecdysone receptor polypeptide to a support such as a plurality of polymeric pins, whereafter the polypeptide resident on the plurality of pins is brought into contact with candidate insecticidal molecules for screening. The molecules being screened may be isotopically labelled so as to permit ready detection of binding. Alternatively, reporter molecules may be utilized which bind to the insect steroid receptor candidate molecule complex. Alternatively, compounds for screening may be bound to a solid support, such as a plurality of pins which are then reacted with the thermostable insect steroid receptor or complex with a partner protein. Binding may, for example, be determined again by isotopic-labelling of the receptor, or by antibody detection or use of another reporting agent.

In an alternative embodiment, insecticidally-active agents are identified using rational drug design, by expressing a USP polypeptide of a N. viridula ecdysone receptor or a fragment thereof which includes the ligand-binding region, optionally in association with a N. viridula EcR or ligand binding domain thereof, and optionally in association with a N. viridula steroid or analogue thereof, so as to form a complex, determining the three-dimensional structure of the ligand binding domain of the complex, and identifying compounds which bind to or associate with the three-dimensional structure of the ligand binding domain, wherein said compounds represent candidate insecticidally-active agents.

The methods described herein for identifying modulators of N. viridula ecdysone receptor and insecticidal compounds, may be performed using prokaryotic or eukaryotic cells, cell lysates or aqueous solutions.

In a seventeenth aspect, the present invention provides a method of identifying a candidate insecticidally-active agent of a N. viridula ecdysone receptor, comprising the steps of

a) expressing the USP polypeptide in combination with an isolated N. viridula EcR polypeptide according to the eleventh aspect so as to form a complex;

b) purifying or precipitating the complex;

c) determining the three-dimensional structure of the ligand binding domain of the complex; and

d) identifying compounds which bind to or associate with the three-dimensional structure of the ligand binding domain, wherein said compounds represent candidate insecticidally-active agents.

Standard procedures are used to determine the three dimensional structure of the receptor polypeptides of the invention, for example using X-ray crystallography and/or nuclear magnetic resonance analysis (see, for example, Bugg et al., 1993; Von Itstein et al., 1993).

Insecticidally-active agents contemplated herein include synthetic chemicals that mimic one or more ligands of N. viridula ecdysone receptor, or the ligand-binding region of said N. viridula ecdysone receptor thereby modulating binding of steroids to said receptor Preferred insecticidally-active agents include compounds which interfere with the binding of ecdysone to N. viridula ecdysone receptor.

A further aspect of this invention accordingly relates to synthetic compounds derived from the three dimensional structure of N. viridula ecdysone receptor polypeptides or N. viridula ecdysone receptor partner protein (USP polypeptide) subunits or fragments thereof, which compounds are capable of binding to said receptors which have the effects of either inactivating the receptors (and thus acting as antagonists) or potentiating the activity of the receptor.

By “derived from” it is meant that the compounds are based on the three dimensional structure of the aforementioned proteins, that is, synthesized to bind, associate or interfere with insect steroid binding or N. viridula ecdysone receptor binding.

The compounds may bind strongly or irreversibly to the ligand binding site or another region of the N. viridula ecdysone receptor or USP and act as agonists or antagonists of insect steroids, or N. viridula ecdysone receptor binding, or otherwise interfere with the binding of ligands, such that ecdysteroids or ecdysone hormones. Such compounds would possess a unique specificity and would have potent insecticidal activity.

In order that the nature of the present invention may be more clearly understood, preferred forms thereof will now be described with reference to the following non-limiting examples.

### EXAMPLE 1

Materials and Methods

1. Experimental Animals and RNA Isolation

N. viridula animals were reared and maintained by the CSIRO Division of Entomology. Fourth instar nymphs were rapidly subjected to total RNA isolation using the guanidine isothiocyanate-CsTFA method (Okayama et al., 1987). mRNA was subsequently purified using the PolA mRNA isolation kit (Promega) and quantitated in aqueous ethidium bromide under UV light.

2. Preparation of a Homologous NvEcR C Domain Screening Probe by PCR

A NvEcR screening probe was amplified from the N. viridula cDNA library (refer to Section 4) employing two degenerate primers to obtain a 138 bp product homologous to the NvEcR DNA binding domain and containing 87 bp of novel NvEcR DNA by the procedure described in Hannan and Hill (1997). The PCR product was cloned into pCR-TOPO2.1 plasmids (TOPO TA Cloning Kit, Invitrogen Life Technologies, Cat. #: K4500-01), and cycle sequenced in both directions (SUPAMAC). The information obtained indicated that the product encoded a steroid nuclear receptor DNA binding domain, and a BLAST search of known sequences indicated highest identity to Leptinotarsa decemlineata (Colorado potato beetle) EcR.

```
TAC CAC TAT AAT GCG CTC ACG TGC GAA GGT TGT AAG
  Y H Y N A L T C E G C K
GGT TTC TTC AGA AGG AGT ATA ATG AAG AAC GCC GTC
  G F F R R S I M K N A V
TAT CAA TGC AAG TAT GGC AAC AAC TGT GAG ATA GAC
  Y Q C K Y G N N C E I D
ATG TAC ATG AGG CGA AAG TGT CAG GAG TGT
  M Y M R R K C Q E C
```

The nucleotide sequence of this novel NvEcR probe is indicated as SEQ ID No: 9 and the amino acid sequence is indicated as SEQ ID No: 10 in the sequence listing.

3. Preparation of a Homologous NvUSP C Domain Screening Probe by PCR

PCR was attempted using both Nv cDNA library and genomic DNA as templates with degenerate primers which have been used in the past to produce USP probes from other organisms (Hannan & Hill, 2001; Tzertzini et al., 1994). However, the PCR failed to generate USP PCR products based on Nezara viridula DNA and rather repeatedly yielded a nucleotide sequence identified by a BLAST search to encode a segment of a NvFTZ-1 protein.

Therefore three separate methods were applied, in parallel, to obtain the NvUSP C domain screening probe.

The first method was based on the finding that the predominant PCR product was a sequence encoding a region within a NvFTZ-1 protein instead of the anticipated NvUSP homologous probe. The Nvftz sequence has a BsrG I site which was absent from the Myzus persicae and Bemisia tabaci USP C domain encoding sequences. Therefore, it was predicted that the desired NvUSP probe sequence would not be digested by BsrG I due to the likely high identity of NvUSP with the known hemipteran USP C domain sequence.

Accordingly, screening of PCR clones in pCR-TOPO2.1 vector by digestion with BsrGI was carried out to eliminate NvFTZ PCR products. However, this technique also failed to isolate a homologous Nezara viridula USP probe as all recombinant PCR clones still appeared to correspond to the FTZ-1 protein.

Therefore, PCR products generated from a genomic DNA template prior to cloning into the pCR-TOPO2.1 vector were digested with BsrGI. The enzyme was then deactivated by incubating the DNA mix at 80° C. for twenty minutes. PCR was then performed on the digested DNA using degenerate primers.

A second PCR method employed was based on the use of the following degenerate primers: H5: 5′-CWS TGG RAA RCA YTA YGG WGT YTA-3′ (SEQ ID No: 11)

(SEQ ID No:12)

H3: 5′-CAT TTY TGA TAY CTR CAR TAY TGR C-3′
where R = A/G, Y = C/T, M = A/C, W = A/T,
S = C/G and K = G/T

The above primers were designed in an attempt to be more specific to the Nvusp sequence based on known usp sequences from Locust migratoria, Myzus persicae and Bemisia tabaci, that were compared to the sequences for Drosophila melanogaster ftz and Nv ftz.

A third PCR method which yielded the first NvUSP probe was a RT PCR method. RT-PCR was performed using Nv total RNA template and two degenerate primers (Tzertzini et al., 1994). A 150 bp probe homologous to NvUSP DNA binding domain was obtained. The 150 bp PCR product included 91 bp of novel NvUSP DNA. The PCR product was cloned into pCR-TOPO2.1 plasmids and cycle sequenced in both directions (SUPAMAC). Analysis of the nucleotide sequence of the 150 bp PCR product indicated that the product encoded a steroid nuclear receptor DNA binding domain. A BLAST search of known sequences indicated that the 150 bp PCR product had the highest nucleotide identity to Riptortus clavatus (Bean Bug) USP.

4. cDNA Library Construction and Screening

A N. viridula cDNA library was constructed from 5 μg of oligo-dT primed mRNA cloned into Lambda ZapII employing a Stratagene kit. The primary library, consisting of 1×10⁶ plaque forming units (pfu) per mL, was amplified once to give a titre of 9.4×10⁸ pfu/mL. Screening for EcR required the plating of 1×10⁶ pfu on an E. coli XL1 Blue (Stratagene) lawn and screening for USP required 2.5×10⁶ pfu. Plaques were lifted onto Hybond N (Amersham) membranes, denatured and fixed according to the manufacturer's instructions. Probes were labelled as described in Hannan and Hill (1997). The membranes were wetted with 2×SSC and incubated with prehybridisation mixture consisting of 42% formamide, 5×SSC, 5× denhardts solution, 0.05 M NaPO₄ (pH 6.4), 0.1% SDS. The radioactively labelled probe and 200 μL of salmon sperm DNA (10 mg/mL) were boiled for 10 min and then rapidly cooled on ice before adding to the prehybridisation mixture. Hybridisation was at 38° C. overnight. The membranes were then washed three times in 2×SSC at room temperature followed by three more washes in 2×SSC at 38° C.

Positive plaques were identified by autoradiography, plaque purified and converted to pBK-CMV phagemids by the excision method (Stratagene) and ORF's were cycle sequenced in both directions. Two EcR isoforms were isolated in, and sequenced from, the clones pBK-CMVEcR 0.1 (NvEcR10) having the nucleotides sequence shown in Sequence ID No: 1 and pBK-CMVEcR11.1 (NvEcR11) having the nucleotides sequence shown in Sequence ID No:2 detailed below. One USP was isolated in the clone pBK-CMVUSP5.1 (NvUSP) and sequenced having the nucleotides sequence shown in Sequence ID No:3 detailed below (refer also to Sequence listing).

Sequence ID No:1
ATGTGCGAGGAGAGCCGGCGGGACGAGTGGACTGTTGGGGGGGGGCAGAA
CGGGTGCCCCTCCCCGACAATGTCCTCCAACAGCTACGAACCCTATAGCC
CCGCTTCAAAGCTAGGTCGAGAGGATCTTTCGCCGGCGAACTCTCTGAAC
GGGTTCTCGGCGGACAGCTGCGATGGCTCCAAGAAGAAGAAGGGGTCGGC
GGCGCGACAGCAGGAGGAGCTCTGCCTGGTCTGTGGTGACAGGGCAAGCG
GTTACCATTATAACGCGCTCACGTGCGAAGGTTGTAAGGGTTTCTTCAGA
AGGAGTATAACGAAGAACGCCGTCTATCAATGCAAGTATGGCAACAACTG
CGAGATAGACATGTACATGAGGCGAAAGTGTCAAGAGTGCAGGCTCAAGA
AATGTCTGTCTGTTGGCATGAGGCCGGAATGTGTTGTACCAGAGTACCAA
TGCGCTGTGAAAAGAAAAGAAAAGAAAGCCCAGAAGGACAAAGATAAACC
AGTCTCGACGACTAATGGTAGCCCTGAATCAATAAAAGTCGAAGAACCAC
ATAGGGTTGTATATAAAACAGAAGGTGAAACTTTGGGCCGACCAGTCCCA
ATTAATGGCGTCAAACCAGTTTCACCCGAACAGGAAGAACTCATCCATAG
GCTAGTGTACTTCCAGAACGAGTACGAGCACCCTTCTGAGGAAGACCTCA
GAAGGATTGTAAGTAATACTCCAAACGAGGATGAAGAGCAGAGTGACCAA
AGGTTCAGGCACCTCACAGAAATCACGATCCTCACAGTACAACTAATAGT
CGAGTTCGCTAAACGGTTACCTGGTTTCGACAAGCTCTTAAGGGAAGATC
AGATTGCTCTTCTCAAGGCCTGTTCAAGTGAGGTGATGATGTTGAGGATG
GCCAGGAAGTACGACGCTACTACAGACTCTATCCTCTTCGCCAACAATCA
GCCCTATAACAGGGAGTCCTATAGCCTAGCAGGCATGGGCGATGTCATTG
AAGACCTGCTTAGGTTTTGTCGACATATGTTCAACATGAAAGTGGACAAT
GCTGAATACGCTCTCCTTACAGCTATTGTCATTTTCTCAGAGAGGCCTGC
ACTAATTGAAGGCTGGAAGGTTGAGAAAATTCAAGAAATATACTTGGAAG
CTTTGAAGTCCTACGTCGATAACAGGTCACGACCAAGATCCCCTACCATA
TTCGCCAAACTGTTGTCTGTTTTGACAGAACTTCGCACGCTAGGCAACCA
AAATTCTGAAATGTGCTTCTCACTCAAGTTGCAAAACAAGAAATTACCCC
CATTCCTTGCAGAAATATGGGACGTCAACACG
Sequence ID No:2
ATGTCCTCCAACAGCTACGAACCCTATAGCCCCGCTTCAAAGCTAGGTCG
AGAGGATCTTTCGCCGGCGAACTCTCTGAACGGGTTCTCGGCGGACAGCT
GCGATGGCTCCAAGAAGAAGAAGGGGTCGGCGGCGCGACAGCAGGAGGAG
CTCTGCCTGGTCTGTGGTGACAGGGCAAGCGGTTACCATTATAACGCGCT
CACGTGCGAAGGTTGTAAGGGTTTCTTCAGAAGGAGTATAACGAAGAACG
CCGTCTATCAATGCAAGTATGGCAACAACTGTGAGATAGACATGTACATG
AGGCGAAAGTGTCAAGAGTGCAGGCTCAAGAAATGTCTGTCTGTTGGCAT
GAGGCCGGAATGTGTTGTACCAGAGTACCAATGCGCTGTGAAAAGAAAAG
AAAAGAAAGCCCAGAAGGACAAAGATAAACCAGTCTCGACGACTAATGGT
AGCCCTGAATCAATAAAAGTCGAAGAACCACATAGGGTTGTATATAAAAC
AGAAGGTGAAACTTTGGGCCGACCAGTCCCAATTAATGGCGTCAAACCAG
TTTCACCCGAACAGGAAGAACTCATCCATAGGCTTGTGTACTTCCAGAAC
GAGTACGAGCACCCTTCTGAGGAAGACCTCAGAAGGATTAATACTCCAAA
CGAGGATGAAGAGCAGAGTGACCAAAGGTTCAGGCACCTCACAGAAATCA
CGATCCTCACAGTACAACTAATAGTCGAGTTCGCTAAACGGTTACCTGGT
TTCGACAAGCTCTTAAGGGAAGATCAGATTGCTCTTCTCAAGGCCTGTTC
AAGTGAGGTGATGATGTTGAGGATGGCCAGGAAGTACGACGCTACTACAG
ACTCTATCCTCTTCGCCAACAATCAGCCCTATAACAGGGAGTCCTATAGC
CTAGCAGGCATGGGCGATGTCATTGAAGACCTGCTTAGGTTTTGTCGACA
TATGTTCAACATGAAAGTGGACAATGCTGAATACGCTCTCCTTACAGCTA
TTGTCATTTTCTCAGAGAGGCCTGCACTAATTGAAGGCTGGAAGGTTGAG
AAAATTCAAGAAATATACTTGGAAGCTTTGAAGTCCTACGTCGATAACAG
GTCACGACCAAGATCCCCTACCATATTCGCCAAACTGTTGTCTGTTTTGA
CAGAACTTCGCACGCTAGGCAACCAAAATTCTGAAATGTGCTTCTCACTC
AAGTTGCAAAACAAGAAATTACCCCCATTCCTTGCAGAAATATGGGACGT
CAACACG
Sequence ID No:3
ATGGAAGCTAATGAGCGAGGACTGAGCTTGGAGAACAACCTGTCGCTGGT
GGGCCCGCAGTCGCCGCTGGACATGAAGCCGGACGCAGCCAGCCTCCTCG
CGGGCAGCTTCAGCCCCTCCCAGGCCTCCAACCCTACAAGCCCTGCGGGA
TTTGGTATGGCACATAACAGTGTACTTGGAAATGGTAACAAGAGCCTGAA
TACCCCTTATCCTCCTAACCATCCACTGAGTGGTTCTAAACATCTCTGCA
GCATATGTGGTGACAGAGCCTCAGGAAAGCATTATGGAGTTTATAGTTGT
GAAGGTTGTAAAGGTTTTTTCAAAAGAACAGTACGTAAAGATTTATCTTA
TGCTTGTCGTGAAGATAAACAATGTCTGGTAGATAAACGTCAAAGGAATA
GATGCCAATATTGCCGATATCAGAAATGTTTATCGATGGGCATGAAAAGG
GAAGCAGTCCAGGAAGAAAGGCAAAGAACTAAAGAGAGAGATCAAAATGA
AGTTGAAAGCACCAGCAGCTTTCACACAGATATGCCTGTTGAAAGGATTT
TAGAAGCAGAAAGAAGAGTTGATTTCAAAGTGGAGCCTATGGTAGAATAT
GAGAATGCAAATACACTTTTTCAAGCGACTGATAAACAACTGGTACAGCT
TGTTGAATGGGCTAAACAAATACCACATTTTACCTCTCTGCCTATCGAAG
ATCAAGTACTCCTTCTTAGAGCTGGTTGGAATGAGCTACTGATAGCAGGA
TTTTCTCATCGCTCTATTGGTGTTAAAGAAAAAATAGTTTTAGGTTCTGG
AGTAACAGTTTGTAGAAATACCGCTCATCAAGCTGGTGTTGATACAATAT
TCGACAGAGCTCTCACTGaATTAGTTTCAAAAATGAGAGAAATGAAGATG
GATAAAGCAGAACTTGGTTGCCTGAGAACTATAATATTATATAATCCAGA
AGTTCGGGGATTGAGGTCAGTAGGAGAAGTTGAAGCTCTGAGGGAAAAAG
TCTATGCATCACTCGAAGAATATACAAGATCAACCCATCCAGAAGAGCCT
GGGCGCTTCGCGAAATTGTTACTTCGACTGCCTTCTCTGAGGTCTATTGG
CCTGAAATGCCTTGAACCACTCTTTTTCTATAGGGTTCTTCATGACATAC
CCATCGATACATTCTTATTACAGATGTTGGAATCATCTGACTTGTCCAAC
AGATTG

5. Amino Acid Sequences of NvEcR10, NvEcR11 and NvUSP Proteins

The conceptually-translated amino acid sequences of NvEcR10 having the amino acid sequence of Sequence ID No: 4 and NvEcR11 having the amino acid sequence of Sequence ID No: 5, are 444 and 419 residues long respectively, and each displays the five domains typical of a nuclear receptor. The NvUSP protein is 402 residues in length having the amino acid sequence of Sequence ID No: 6 and also displays all domains typical of a nuclear receptor.

Sequence ID No:4
M C E E S R R D E W T V G G G Q N G C P S P T M S
S N S Y E P Y S P A S K L G R E D L S P A N S L N
G F S A D S C D G S K K K K G S A A R Q Q E E L C
L V C G D R A S G Y H Y N A L T C E G C K G F F R
R S I T K N A V Y Q C K Y G N N C E I D M Y M R R
K C Q E C R L K K C L S V G M R P E C V V P E Y Q
C A V K R K E K K A Q K D K D K P V S T T N G S P
E S I K V E E P H R V V Y K T E G E T L G R P V P
I N G V K P V S P E Q E E L I H R L V Y F Q N E Y
E H P S E E D L R R I V S N T P N E D E E Q S D Q
R F R H L T E I T I L T V Q L I V E F A K R L P G
F D K L L R E D Q I A L L K A C S S E V M M L R M
A R K Y D A T T D S I L F A N N Q P Y N R E S Y S
L A G M G D V I E D L L R F C R H M F N M K V D N
A E Y A L L T A I V I F S E R P A L I E G W K V E
K I Q E I Y L E A L K S Y V D N R S R P R S P T I
F A K L L S V L T E L R T L G N Q N S E M C F S L
K L Q N K K L P P F L A E I W D V N T
Sequence ID No:5
M S S N S Y E P Y S P A S K L G R E D L S P A N S
L N G F S A D S C D G S K K K K G S A A R Q Q E E
L C L V C G D R A S G Y H Y N A L T C E G C K G F
F R R S I T K N A V Y Q C K Y G N N C E I D M Y M
R R K C Q E C R L K K C L S V G M R P E C V V P E
Y Q C A V K R K E K K A Q K D K D K P V S T T N G
S P E S I K V E E P H R V V Y K T E G E T L G R P
V P I N G V K P V S P E Q E E L I H R L V Y F Q N
E Y E H P S E E D L R R I N T P N E D E E Q S D Q
R F R H L T E I T I L T V Q L I V E F A K R L P G
F D K L L R E D Q I A L L K A C S S E V M M L R M
A R K Y D A T T D S I L F A N N Q P Y N R E S Y S
L A G M G D V I E D L L R F C R H M F N M K V D N
A E Y A L L T A I V I F S E R P A L I E G W K V E
K I Q E I Y L E A L K S Y V D N R S R P R S P T I
F A K L L S V L T E L R T L G N Q N S E M C F S L
K L Q N K K L P P F L A E I W D V N T
Sequence ID No:6
M E A N E R G L S L E N N L S L V G P Q S P L D M
K P D A A S L L A G S F S P S Q A S N P T S P A G
F G M A H N S V L G N G N K S L N T P Y P P N H P
L S G S K H L C S I C G D R A S G K H Y G V Y S C
E G C K G F F K R T V R K D L S Y A C R E D K Q C
L V D K R Q R N R C Q Y C R Y Q K C L S M G M K R
E A V Q E E R Q R T K E R D Q N E V E S T S S F H
T D M P V E R I L E A E R R V D F K V E P M V E Y
E N A N T L F Q A T D K Q L V Q L V E W A K Q I P
H F T S L P I E D Q V L L L R A G W N E L L I A G
F S H R S I G V K E K I V L G S G V T V C R N T A
H Q A G V D T I F D R A L T E L V S K M R E M K M
D K A E L G C L R T I I L Y N P E V R G L R S V G
E V E A L R E K V Y A S L E E Y T R S T H P E E P
G R F A K L L L R L P S L R S I G L K C L E P L F
F Y R V L H D I P I D T F L L Q M L E S S D L S N
R L

6. Characterization of Various Domains and Helices of NvEcR10, NvEcR11 and NvUSP Protein

Based on the full length nucleotide and amino acid sequences of N. viridula NvEcR10, NvEcR11 and NvUSP detailed at sections 4 and 5, various domains and helices of the proteins have been defined in Tables 1 and 2.

TABLE 1
Nucleotide and amino acid sequences corresponding to various
domains and helices of N. viridula NvEcR10 and NvEcR1

| | Nucleotides of | | Amino acids of | |
|---|---|---|---|---|
| | SEQ ID | SEQ ID | SEQ ID | SEQ ID |
| Domain(s)/ | No: 1 | No: 2 | No: 4 | No: 5 |
| Helices | Nv EcR10 | NvEcR11 | NvEcR10 | NvEcR11 |
| A/BCDE | 1-1332 | 1-1257 | 1-444 | 1-419 |
| A/B | 1-222 | 1-153 | 1-74 | 1-51 |
| C | 223-420 | 154-351 | 75-140 | 52-117 |
| D | 421-615 | 352-646 | 141-205 | 118-182 |
| E | 616-1332 | 647-1257 | 206-444 | 183-419 |
| CDE | 223-1332 | 154-1257 | 75-444 | 52-419 |
| DE | 421-1332 | 352-1257 | 141-444 | 118-419 |
| Helices | 631-1326 | 572-1251 | 211-442 | 188-417 |
| 1-12 | | | | |

TABLE 2
Nucleotide and amino acid sequences corresponding
to various domains and helices of NvUSP protein

| | Nucleotides of | Amino acids of |
|---|---|---|
| Domain(s)/ | SEQ ID No: 3 | SEQ ID No: 6 |
| Helices | Nv USP | NvUSP |
| A/BCDE | 1-1206 | 1-402 |
| A/B | 1-246 | 1-82 |
| C | 247-444 | 83-148 |
| D | 445-516 | 149-172 |
| E | 517-1206 | 173-402 |
| CDE | 247-1206 | 83-402 |
| DE | 445-1206 | 149-402 |
| Helices 1-12 | 538-1182 | 180-394 |

7. NvEcR10 and NvEcR11 Isoforms

NvEcR10 and NvEcR11 differ at their N-termini, (A/B Domain), as well as in their E Domains. The difference in sequence seen in A/B domain of these two EcR variants is due to the use of different translational initiation sites (FIG. 1). NvEcR10 contains two additional amino acid residues (ValSer) in Domain E (at positions 237 and 238 of SEQ ID NO:4). Sequence analysis of the protein isoforms confirmed that the NvEcR10 isoform contains two additional amino acid residues (ValSer) in the E domain as indicated in the following tryptic digest peptide:

| | |
|---|---|
| IVSNTPNEDEEQSDQR | (SEQ ID No:7) |

The corresponding tryptic peptide from NvEcR11 protein is as follows:

| | |
|---|---|
| INTPNEDEEQSDQR | (SEQ ID No:8) |

The peptides were identified by MALDI-TOF/TOF in the Australian Proteome Analysis Facility.

In order to investigate whether this addition/subtraction of sequence is due to different genes or alternative splicing, at least 10 individual adult and nymph N. viridula from a heterogenous population were used for separate genomic DNA isolations.

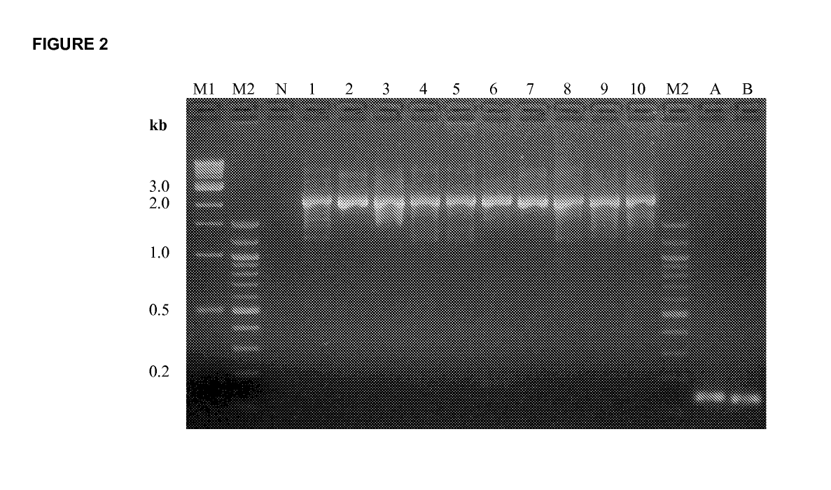

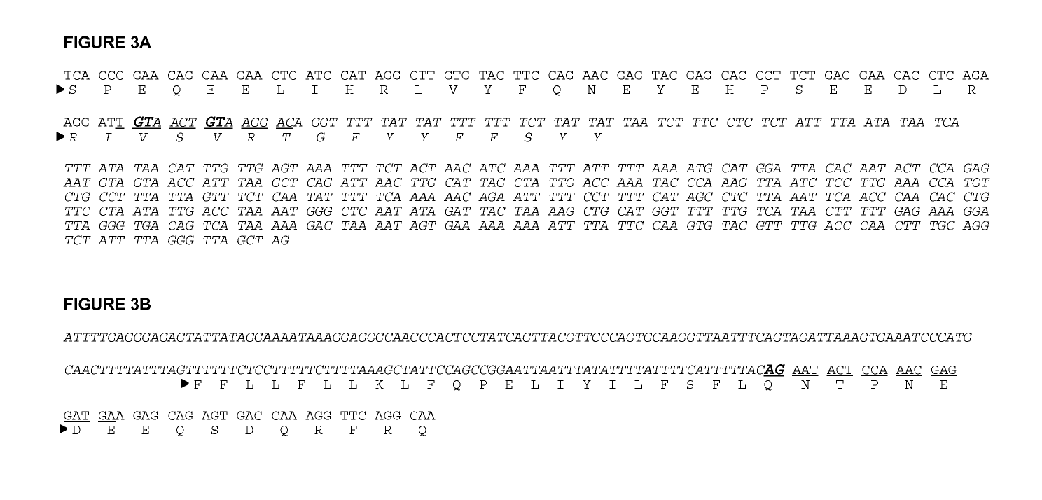

PCR using primers flanking the area where the cDNA for NvEcR10 and NvEcR11 E domains differ was performed using both genomic and cDNA templates. All genomic DNA templates yielded products of roughly 2.5 kb in size whereas the cDNA yielded products of expected size, below 200 bp in length (FIG. 2). This suggested the presence of an intron. Sequence analysis of the 2.5 kb PCR products using three different splice site predictor programs confirmed the presence of two closely positioned donor sites and an acceptor site (FIG. 3) which would account for the difference in the E domains of NvEcR10 and NvEcR11.

Splice sites which give rise to NvEcR10 and NvEcR11 all score as high, if not higher than known corresponding splice sites of DmEcR, Aedes aegypti EcR (AeEcR), and Anolopheles gambiae (AgEcR) (Table 3).

TABLE 3
A comparison of scores from 3 different splice site predictor programs performed on genomic
DNA sequence from NvEcR, three other EcR receptors from insects and the orthologs from
human, HsRAR α, HsLXRα1&3. The donor site scores for NvEcR10 and NvEcR11 are
indicated in brackets by (10) and (11) respectively. Likewise, the acceptor site scores
for HsLXRα1 and HsLXRα3 are denoted in brackets by (1) and (3) respectively. D =
Donor site, A = Acceptor site, Dm = Drosophila melanogaster, Hs = Homo sapiens,
ASSP = Alternative Splice Site Predictor, NR = not recognised. Ag = Anopheles
gambiae. Ae = Aedes aegypti.

| Splice Site | | Consensus | | | | | | |
|---|---|---|---|---|---|---|---|---|
| Predictor | Splice | Sequence | | | | | | |
| Program | Signal | Database | NvEcR | DmEcR | AgEcR | AeEcR | HsRARα | HsLXRα |
| NEURAL | D | Dm | 0.70 (10) | 0.81 | 0.77 | 0.49 | 0.60 | 0.13 |
| NETWORK | | | NR (11) | | | | | |
| | | Hs | NR (10) | 0.45 | 0.95 | 0.93 | 0.89 | 0.27 |
| | | | 0.98 (11) | | | | | |
| | A | Dm | 0.99 | 0.94 | 0.97 | 0.98 | 0.75 | 0.32 (1) |
| | | | | | | | | 0.65 (3) |
| | | Hs | 0.99 | 0.96 | 0.99 | 0.99 | 0.95 | 0.20 (1) |
| | | | | | | | | 0.79 (3) |
| SPLICEVIEW | D | Dm | 0.80 (10) | 0.86 | 0.86 | 0.83 | 0.78 | 0.75 |
| | | | 0.84 (11) | | | | | |
| | | Hs | 0.80 (10) | 0.82 | 0.82 | 0.80 | 0.79 | 0.76 |
| | | | 0.82 (11) | | | | | |
| | A | Dm | 0.94 | 0.87 | 0.85 | 0.85 | 0.81 | NR (1) |
| | | | | | | | | 0.76 (3) |
| | | Hs | 0.89 | 0.86 | 0.86 | 0.88 | 0.93 | 0.80 (1) |
| | | | | | | | | 0.83 (3) |
| ASSP | D | Hs | 7.83 (10) | 9.05 | 10.19 | 7.86 | 7.78 | 5.37 |
| | | | 9.94 (11) | | | | | |
| | A | Hs | 11.35 | 8.85 | 8.76 | 8.41 | 11.11 | 3.63 (1) |
| | | | | | | | | 6.39 (3) |

### EXAMPLE 2

Construction of a Baculovirus for Co-Expression of the Ligand Binding Regions of NvEcR and NvUSP

Step 1A: Cloning pCR-TOPO2.1 His₆EcR_{DEF}

Two primers were designed and synthesized so that the DEF domains of both EcR isoforms could be subcloned into separate pCR-TOPO2.1 plasmids for subsequent cloning into pFastBacDual plasmids.

Primer 1 (SEQ ID No:13): This forward primer has an incorporated Not I site followed by a hexa-HIS tag and the very first D domain sequence which is shared by both pBK-CMVEcR10.1 and pBK-CMVEcR11.1. SEQ ID No: 14 represents the protein sequence.

| | NotI |
|---|---|
| 1241 | GCGGCCGC ATG GGT ATG AGA GGA TCG CAT CAC CAT |
| | 1 M G M R G S H H H |
| 1276 | CAC CAT CAC AGG CCG GAA TGT GTT GTA CCA |
| 10 | H H H R P E C V V P |

Primer 2 (SEQ ID No:15): The reverse primer has an incorporated Xba I site followed by sequence complementary to the 3′ end of the DEF domains of both pBK-CMVEcR10.1 and pBK-CMVEcR11.1.

| | XbaI |
|---|---|
| 2134 | TCTAGAGC TTC TCA CGT GTT GAC GTC CCA |

Using pBK-CMVEcR10.1 and pBK-CMVEcR11.1 as templates, and Pwo DNA Polymerase (Roche, Cat #: 1644947), the DEF region of each respective clone alone, was amplified by PCR. The PCR protocol involved incubation at 94° C. for 2 minutes followed by 5 cycles of PCR as follows: 94° C. for 15 s, 45° C. for 30 s, 72° C. for 1 min, followed by 12 cycles of PCR at 94° C. for 15 s, 60° C. for 30 s, 72° C. at 1 min. Then a final elongation step at 72° C. for 5 min was performed. After PCR products of the correct size were confirmed by agarose gel electrophoresis, Taq polymerase was added to the PCR mix and incubated for 9 min @ 72 degrees. This facilitated the production of a deoxyadenosine (A) 3′ overhang on the PCR products allowing for ligation to the linearised pCR-TOPO2.1 plasmid which has a single overhanging 3′ deoxythymidine (T) residue. Ligation of respective PCR products and pCR-TOPO2.1 plasmids produces the recombinant plasmids pCR-TOPO2.1 HiS₆EcR_{DEF}10.1 and pCR-TOPO2.1 HiS₆EcR_{DEF}11.1.

Step 1B: Cloning pCR-TOPO2.1 FLAG-USP_{DEF}

Using an identical technique to that in step 1A, primers 3 and 4 were used to subclone the DEF region of NvUSP into pCR-TOPO2.1 from the template pBK-CMVUSP5.1, producing the recombinant plasmid pCR-TOPO2.1 FLAG USP_{DEF}5.1.

Primer 3 (SEQ ID No:16): This forward primer has an incorporated Nco I site 5 prime of the FLAG immunoaffinity tag followed by the very first D domain sequence in pBK-CMVUSP5.1. SEQ ID NO:17 represents the amino acid sequence.

| | NcoI |
|---|---|
| 606 | CC ATG GCAGAC TAC AAG GAC GAC GAT GAC AAG AAA AGG GAA |
| | 1 M A D Y K D D D D K K R E |
| 647 | GCA GTC CAG GAA |
| 14 | A V Q E |

Primer 4 (SEQ ID NO:18): This reverse primer was designed such that the PCR product would have an incorporated Kpn I site 3 prime to the end. of the DEF encoding sequence.

| | KpnI |
|---|---|
| 1353 | GGTACCCC AAG CTA CAA TCT GTT GGA CAA |

Step 2: Cloning pFastBacDual FLAG USP_{DEF}

pCR-TOPO2.1 FLAG USP_{DEF}5.1 was digested with Kpn I and Not I. The resultant fragments were ligated into pFastBacDual (Invitrogen) which had also been digested with Kpn I and Not I and treated with phosphatase by standard methods. Restriction digest followed by cycle sequencing of both strands, confirmed the correct sequence for recombinant plasmid pFastBacDual FLAG USP_{DEF}.

Step 3: Cloning pFastBacDual His₆ EcR_{DEF} FLAG USP_{DEF}, clones NvEcR10 and NvEcR11.

Recombinant plasmids, pFastBacDual FLAG USP_{DEF}, pCR-TOPO2.1 His₆EcR_{DEF}10.1 and pCR-TOPO2.1 HiS₆EcR_{DEF}11.1, were digested with Not I and treated with phosphatase following standard methods. Two separate ligations were performed; one with the resultant fragment from pCR-TOPO2.1 His₆EcR_{DEF}10.1 and the linearised plasmid pFastBacDual

FLAG USP_{DEF} which were treated to construct the recombinant plasmid pFastBacDual His₆ EcR_{DEF} FLAG USP_{DEF}10; and the other with the resultant fragment of pCR-TOPO2.1 HiS₆EcR_{DEF}11.1 and the linearised plasmid pFastBacDual FLAG USP_{DEF} which were also treated to construct the recombinant plasmid pFastBacDual His₆ EcR_{DEF} FLAG USP_{DEF}11.

All digested plasmids and inserts in steps 2 and 3 were isolated using gel electrophoresis with crystal violet dye instead of ethidium bromide, this was to minimise possible mutation which may have resulted from the use of ethidium bromide in preparatory gels.

Step 4. Transposition from pFastBacDual His₆ EcR_{DEF} FLAG USP_{DEF} into a Bacmid and Baculovirus Construction.

The mini-Tn7 expression cassette of both donor plasmids pFastBacDual His₆ EcR_{DEF} FLAG USP_{DEF}, clones NvEcR10 and NvEcR11 were transposed, in parallel, into a baculovirus genome by transformation into DH10Bac competent cells and selection of white colonies. White colonies were colony purified and grown up in liquid culture. Minipreparations of bacmid DNA were made using an alkaline lysis procedure in which attention was payed to minimisation of shear forces. The resultant DNA was monitored for the presence of high molecular weight bacmid DNA by electrophoresis through a 0.5% agarose gel.

Mid-log phase Sf9 insect cells were transfected with bacmid DNA using standard procedures and grown at 26-28° C. until signs of infection were apparent, i.e. decrease in cell growth, increase in cell diameter and detachment. Primary virus was harvested from the culture supernatant. This primary virus stock was then amplified twice, first at small scale (2 mL) to determine the best MOI, and then in 30-50 mL shaker cultures in order to have enough high titre viruses to produce the respective recombinant proteins for further analyses.

### EXAMPLE 3

Production and Purification of Recombinant Heterodimeric EcR/USP Ligand Binding Regions

Medium scale production of recombinant heterodimeric NvEcR-NvUSP ligand binding regions for both clones NvEcR10 and NvEcR11 was achieved by infection with amplified virus of suspension cultures of Sf9 insect cells in Schott bottles in a shaker platform incubator maintained at 26-28° C. Insect cells infected with the virus engineered to express NvEcR-NvUSP ligand binding regions were shown by gel electrophoresis and western blotting to contain the expressed EcR_{DEF} and USP_{DEF} polypeptides tagged by (His)₆ and FLAG respectively. The expressed ligand binding region heterodimers for clones NvEcR10 and NvEcR11 were named NvEcR10_{DEF}_{^{−}}NvUSP_{DEF} and NvEcR11_{DEF}_{^{−}}NvUSP_{DEF} respectively.

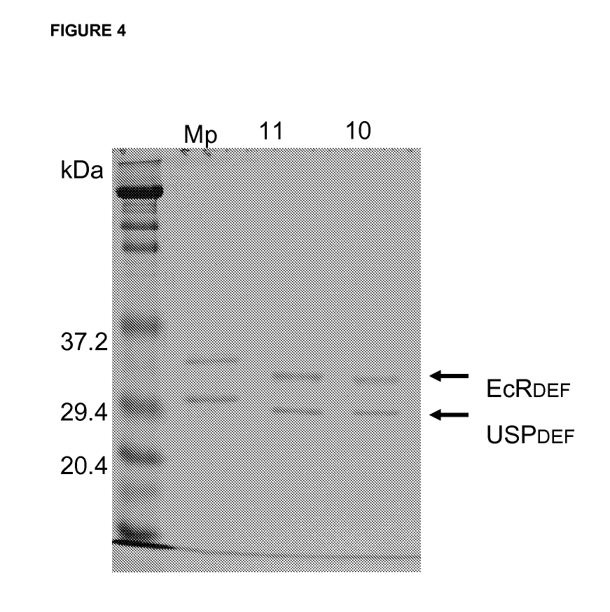

Successful 1L cultures yielded 6 g wet cells, which contained approximately 1 to 1.5 mg recombinant protein per gram of cells. Heterodimer could be affinity-purified from cell extracts by using a nickel chelate resin (IMAC, immobilized metal affinity chromatography) to capture the His₆-tag of the recombinant EcR ligand binding region, followed by elution with an imidazole-containing buffer. Yields were estimated from measurements of protein concentration and from binding of [³H]-ponasterone A. Identity, integrity and purity were monitored by SDS-polyacrylamide gel electrophoresis (see FIG. 4), MALDI TOF-TOF, and amino acid analysis.

Ligand Binding Studies

Ligand binding was measured by a [³H]ponasterone A binding assay which involved trapping of [³H]ponasterone A-receptor protein complex on glass fibre discs and washing away unbound steroid before scintillation counting in a Packard Tri-carb scintillation analyser. All such assays, unless otherwise stated, were conducted at the same concentration of bindable [³H]ponasterone A (2.2 nM, after correcting for the fraction of radioactive material that could never be bound no matter how much receptor was added). Binding by the receptor was expressed in terms of arbitrary units (U) that, in the linear range of the assay (i.e. up to 30 000 cpm bound per filter), were directly proportional to filter-bound cpm. Historically, IU was defined as the amount of [³H]ponasterone A binding conveyed by a fixed volume of a specific receptor preparation. Differences in the assay over time were corrected for empirically by re-assaying the reference receptor preparation with each new batch of [³H]ponasterone A and calculating a batch-specific adjustment that was incorporated into the numerical factor used to convert filter-bound cpm into ligand binding units. On average, 1 pmol of receptor-bound [³H]ponasterone A corresponds to approximately 15.2 U. Specific activity values (U/μg protein) can be calculated for different preparations of purified receptor and these reflect the purity of the preparation, the proportion of receptor that is functional, and the Kd value for that particular receptor.

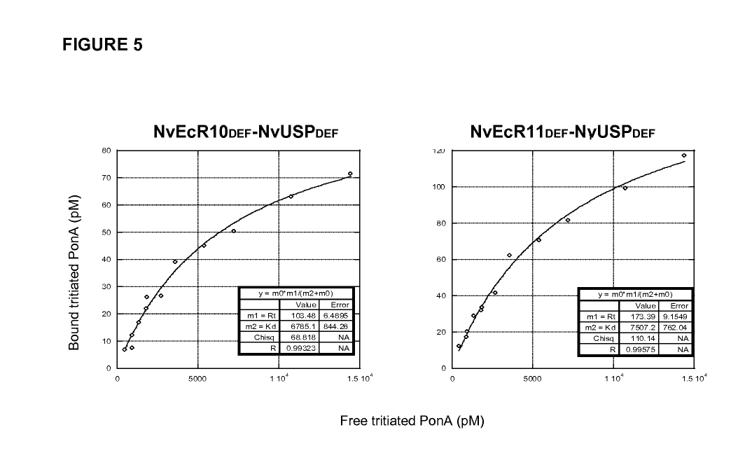

The recombinant cell lysates had a greatly enhanced ability to bind the radiolabelled ecdysteroid, [³H]ponasterone A, compared to control cells. These results indicated that the recombinant virus was expressing functional ligand binding regions that were able to heterodimerise and form a recombinant N. viridula receptor ligand binding region that bound ecdysteroids with high affinity. Equilibrium binding studies with [H]ponasterone A as ligand gave a K_{d} value of 6.8±0.8 nM for NvEcR10_{DEF}_{^{−}}NvUSP_{DEF} clone and 7.5±0.8 nM for NvEcR11_{DEF}_{^{−}}NvUSP_{DEF} clone (FIG. 5). Purified preparations of these recombinants have lower specific activity than those previously studied by this group (Table 4) and this is consistent with the observation of higher Kd values for clones NvEcR10_{DEF}_{^{−}}NvUSP_{DEF} and NvEcR11_{DEF}_{^{−}}NvUSP_{DEF} in equilibrium binding studies (FIG. 5). Taken together, these results suggest that the Nv recombinants may have a slightly lower affinity for ponasterone A.

TABLE 4
Comparison of specific activity and Kd values for recombinant
ligand binding domains (DEF regions) across ecdysone receptors
from five different insect species.

| IMAC-purified recombinant | Specific | |
|---|---|---|
| ligand binding region | activity | Kd |
| heterodimers | (U/μg) | (nM) |
| Nezara viridula, | 5.0 | 6.8 ± 0.8 |
| NvEcR10_{DEF}-NvUSP_{DEF} | | |
| Nezara viridula, | 7.3 | 7.5 ± 0.8 |
| NvEcR11_{DEF}-NvUSP_{DEF} | | |
| Lucilia cuprina | 15.4-24.3 | 1.0 ± 0.1 |
| Myzus persicae | 17.3-18.8 | 0.7 ± 0.1 |
| Bemisia tabaci | 11.3 | 1.2 ± 0.2 |
| Helicoverpa armigera | — | 2.5 ± 0.1 |

### EXAMPLE 4

Competition Binding Studies

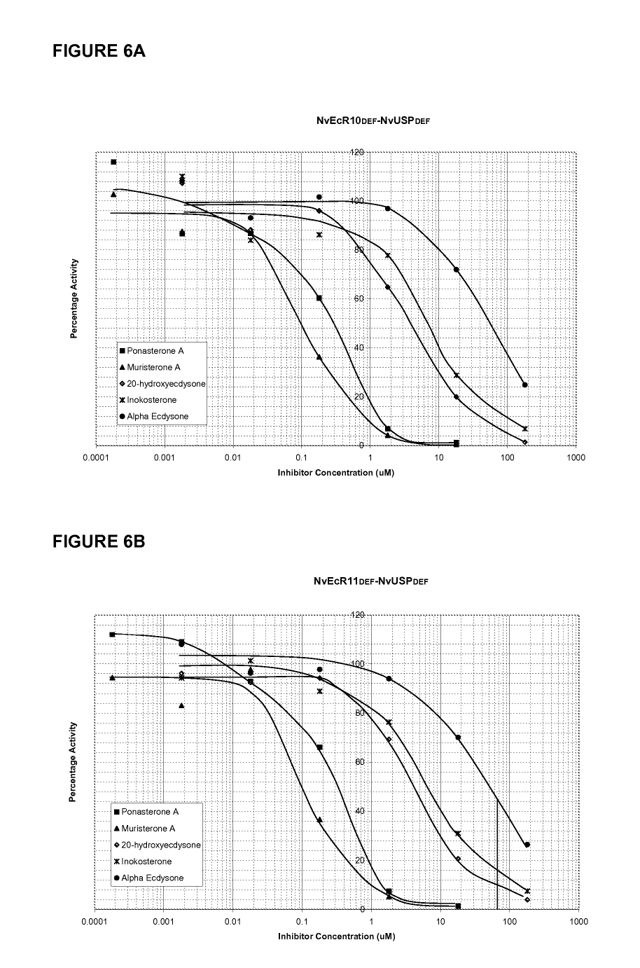

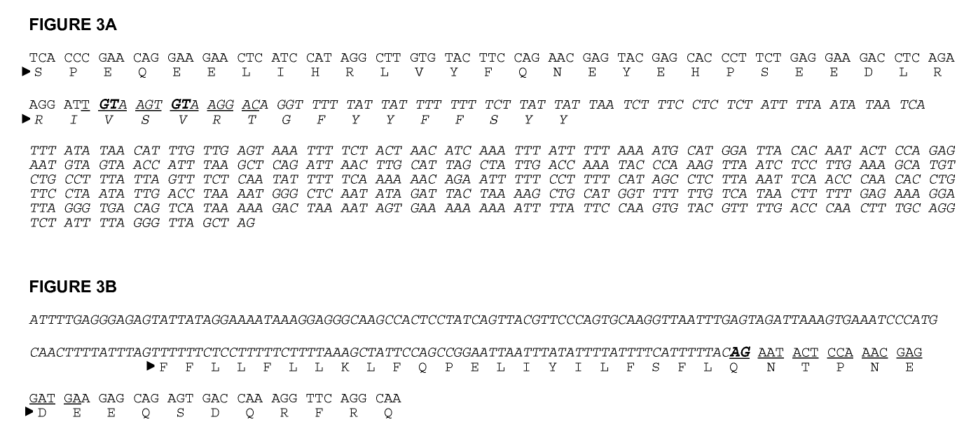

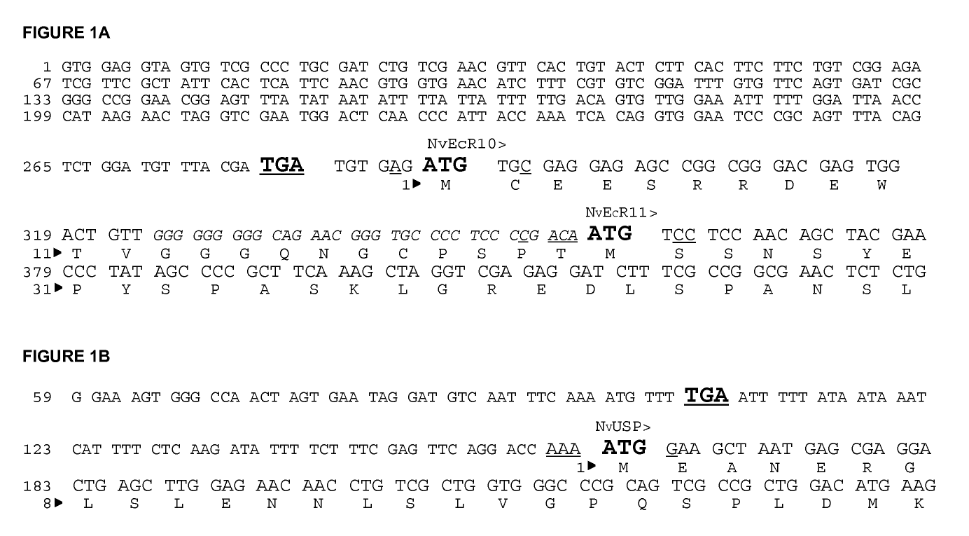

Increasing concentrations of, ponasterone A, muristerone A, 20-hydroxyecdysone, inokosterone, and α-ecdysone (2β, 3β, 14α,22R, 25-pentahydroxy-5β-cholest-7-ene-6-one) were used to study the ability to compete with a 1.3 nM bindable concentration of [³H] ponasterone A for binding to the Nv recombinant heterodimers NvEcR10_{DEF}_{^{−}}NvUSP_{DEF} and NvEcR11_{DEF}_{^{−}}NvUSP_{DEF}. IC₅₀ values were obtained from smooth curves drawn through the data points (see FIG. 6), and from these Ki values were calculated using the Cheng-Prusoff equation (Cheng et at, 1973). The results (see Table 5) show NvEcR10_{DEF}_{^{−}}NvUSP_{DEF} and NvEcR11_{DEF}_{^{−}}NvUSP_{DEF} to have almost identical profiles in regards to competitive inhibition of the studied compounds, muristerone A (Ki 0.1 μM) being the most effective and α-ecdysone being the least effective competitor (Ki 45.2-46.2 μM). Differences in competitive inhibition data for the ecdysteroids tested were not found to be statistically significant between NvEcR10_{DEF}_{^{−}}NvUSP_{DEF} and NvEcR11_{DEF}_{^{−}}NvUSP_{DEF}.

TABLE 5
IC50 and Ki values
The two heterodimers NvEcR10_{DEF}-NvUSP_{DEF} and
NvEcR11_{DEF}-NvUSP_{DEF} present with IC₅₀ and Ki values which are not
statistically significantly different.

| | NvEcR10_{DEF}-NvUSP_{DEF} | | NvEcR11_{DEF}-NvUSP_{DEF} | |
|---|---|---|---|---|
| | IC₅₀ (μM) | Ki (μM) | IC₅₀ (μM) | Ki (μM) |
| Ponasterone A | 0.26 | 0.2 | 0.28 | 0.2 |
| Muristerone A | 0.12 | 0.1 | 0.12 | 0.1 |
| 20-hydroxyecdysone | 4 | 3.6 | 4.5 | 3.8 |
| Inokosterone | 7.5 | 6.3 | 8 | 6.8 |
| α-Ecdysone | 55 | 46.2 | 53 | 45.2 |

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

Ausubel F M et. al (1992) Current Protocols in Molecular Biology, Wiley, New York.
Barclay M V L. (2004) The Entomologist's Records and Journal of Variation, 116: 55-58
Bugg C E, Carson W M, Montgomery J A. (1993) Scientific American, 269(6): 60-66.
Cheng Y and Prusoff W H. (1973) Biochem. Pharmacol. 22, 3099-3108
Cho W L, Kapitskaya M Z, Raikhel A S. (1995) Insect Biochem. Molec. Biol. 25:19-27.
Devereux J, Haeberli P, Smithies O. (1984). Nucl. Acids Res. 12: 387-395.
Dhadialla T S, Carlson G R, Le D P. (1998), Annu. Rev. Entomol. 43: 545-569.
Gluzman Y and Shenk T. (1983) Enhancers and Eukaryotic Gene Expression, Cold Spring Harbor Press, New York.
Hannan G N and Hill R J. (1997) Insect Biochem. Mol. Biol. 27: 479-488.
Hannan, G N and Hill R J. (2001) Insect Biochem Mol. Biol. 31(8):771-81.
Koelle M R, et. al. (1991) Cell. 67(1):59-77.
Lafont R and Dinan L. (2003) J Insect Sci. 3:7.
Liu Q and Xue Q. (2005) Journal of Genetics 84(3): 317-322.
Needleman S B and Wunsch C D. (1970) J Mol. Biol. 48: 443-453.
Okayama H et. al. (1987) Methods Enzymol. 154:3-28.
Padidam M, et. al. (2003) Transgenic Res. 12(1):101-9.
Potrykus I. (1990) BioTechnology 8: 535-542.
Riddiford L M, Cherbas P, Truman J W. (2000) Vitam Horm, 60:1-73.
Saleh et. al. (1998) Mol Cell Endocrinol. 143: 91-99.
Sambrook J, Fritsch E F, Maniatis T. Molecular Cloning: A Laboratory Manual, Second Edition, Cold Spring Harbor Laboratory Press, Cold Spring Harbor (1989)
Swevers L, et. al. (1995) Insect Biochem Mol Biol. 25(7):857-66.
Thompson J D, Higgins D G, Gibson T J. (1994) Nuc. Acids Res. 22: 4673-4680.
Tzertzinis G, Malecki A, Kafatos F C. (1994) J Mol Biol. 238(3):479-86.
Verras M, et. al. (1999) Eur J Biochem. 265: 798-808.
Von Itzstein M, et. al. (1993) Nature 363(6428): 418-423

1. An isolated polynucleotide comprising a nucleotide sequence which encodes or is complementary to a sequence which encodes a Nezara viridula EcR polypeptide, wherein the polynucleotide comprises a nucleic acid sequence at least 95% identical to the sequence set forth in SEQ ID No:1 or SEQ ID No:2.

2. An isolated polynucleotide according to claim 1, wherein the polynucleotide comprises the sequence set forth in SEQ ID No:1 or SEQ ID No:2.

3. An isolated polynucleotide comprising a nucleotide sequence which encodes or is complementary to a sequence which encodes a USP polypeptide of a N. viridula ecdysone receptor, wherein the polynucleotide comprises a nucleic acid sequence at least 95% identical to the sequence set forth in SEQ ID No:3.

4. An isolated polynucleotide according to claim 3, wherein the polynucleotide comprises the sequence set forth in SEQ ID No:3.

5. An isolated polynucleotide comprising a nucleotide sequence which encodes or is complementary to a sequence which encodes a N. viridula EcR polypeptide, the polypeptide comprising an amino acid sequence at least 95% identical to the sequence set forth in SEQ ID No:4 or SEQ ID No:5.

6. An isolated polynucleotide according to claim 5, wherein the encoded polypeptide comprises the amino acid sequence set forth in SEQ ID No:4 or SEQ ID No:5.

7. An isolated polynucleotide comprising a nucleotide sequence which encodes or is complementary to a sequence which encodes a USP polypeptide of a N. viridula ecdysone receptor, the polypeptide comprising an amino acid sequence at least 95% identical to the sequence set forth in SEQ ID No:6.

8. An isolated polynucleotide according to claim 7, wherein the encoded polypeptide comprises the amino acid sequence set forth in SEQ ID No:6.

9. An isolated polynucleotide which encodes an N. viridula EcR polypeptide wherein the polynucleotide has a sequence that hybridises under high stringency conditions to the nucleotide sequence set forth in SEQ ID No:1 or SEQ ID No:2, or a sequence fully complementary thereto, wherein high stringency conditions are a hybridisation and/or wash carried out in less than the ionic strength of 5×SSC, 0.05 M sodium phosphate, 42% formamide, 0.1% SDS at a temperature of at least 38° C. and a washing step of at least 38° C. in 2×SSC.

10. An isolated polynucleotide which encodes a USP polypeptide of a N. viridula ecdysone receptor wherein the polynucleotide has a sequence that hybridises under high stringency conditions to the nucleotide sequence set forth in SEQ ID No:3, or to a sequence fully complementary thereto, wherein high stringency conditions are a hybridisation and/or wash carried out in less than the ionic strength of 5×SSC, 0.05 M sodium phosphate, 42% formamide, 0.1% SDS at a temperature of at least 38° C. and a washing step of at least 38° C. in 2×SSC.

11. An isolated EcR polypeptide of the N. viridula ecdysone receptor comprising an amino acid sequence at least 95% identical to the sequence set forth in SEQ ID No:4 or SEQ ID No:5.

12. An isolated EcR polypeptide according to claim 11 comprising the amino acid sequence set forth in SEQ ID No: 4 or SEQ ID No:5.

13. An isolated USP polypeptide of the N. viridula ecdysone receptor comprising an amino acid sequence at least 95% identical to the sequence set forth in SEQ ID No:6.

14. An isolated USP polypeptide according to claim 13 comprising the amino acid sequence set forth in SEQ ID No:6

15. An isolated fragment of the N. viridula EcR polypeptide according to claim 11, wherein the fragment comprises one or more functional domain regions.

16. An isolated fragment of the N. viridula USP polypeptide according to claim 13, wherein the fragment comprises one or more functional domain regions.

17. An isolated EcR-USP heterodimer comprising an EcR polypeptide of the N. viridula ecdysone receptor set forth in SEQ ID No:4 or SEQ ID No:5 or ligand binding domain thereof in association with an EcR partner protein (USP polypeptide) set forth in SEQ ID No:6 or ligand binding domain thereof.

18. A method of identifying a modulator of a N. viridula ecdysone receptor comprising:
(a) assaying the binding of a reporter ligand to a N. viridula ecdysone receptor polypeptide according to claim 11 in the presence of a potential modulator; and
(b) assaying the binding of a reporter ligand to the ecdysone receptor polypeptide according to claim 11 without said potential modulator; and
(c) comparing the binding of the reporter ligand in the presence of the potential modulator to the binding of the reporter ligand in the absence of the potential modulator,
wherein a difference in the level of binding indicates that said potential modulator is a modulator of the N. viridula ecdysone receptor.

19. A method of identifying a modulator of a N. viridula ecdysone receptor comprising:
(a) assaying the binding of a reporter ligand to a N. viridula ecdysone receptor polypeptide according to claim 13 in the presence of a potential modulator; and
(b) assaying the binding of a reporter ligand to the ecdysone receptor polypeptide according to claim 13 without said potential modulator; and
(c) comparing the binding of the reporter ligand in the presence of the potential modulator to the binding of the reporter ligand in the absence of the potential modulator,
wherein a difference in the level of binding indicates that said potential modulator is a modulator of the N. viridula ecdysone receptor.


SEQUENCE LISTING (26 sequences)

SEQ ID NO: 1 - DNA, 1332 bp, Artificial
  Nezara viridula clone pBK-CMVEcR10.1 (NvEcR10)
atgtgcgaggagagccggcgggacgagtggactgttgggggggggcagaacgggtgcccctccccgacaatgtcctccaacagctacgaaccctatagccccgcttcaaagctaggtcgagaggatctttcgccggcgaactctctgaacgggttctcggcggacagctgcgatggctccaagaagaagaaggggtcggcggcgcgacagcaggaggagctctgcctggtctgtggtgacagggcaagcggttaccattataacgcgctcacgtgcgaaggttgtaagggtttcttcagaaggagtataacgaagaacgccgtctatcaatgcaagtatggcaacaactgcgagatagacatgtacatgaggcgaaagtgtcaagagtgcaggctcaagaaatgtctgtctgttggcatgaggccggaatgtgttgtaccagagtaccaatgcgctgtgaaaagaaaagaaaagaaagcccagaaggacaaagataaaccagtctcgacgactaatggtagccctgaatcaataaaagtcgaagaaccacatagggttgtatataaaacagaaggtgaaactttgggccgaccagtcccaattaatggcgtcaaaccagtttcacccgaacaggaagaactcatccataggctagtgtacttccagaacgagtacgagcacccttctgaggaagacctcagaaggattgtaagtaatactccaaacgaggatgaagagcagagtgaccaaaggttcaggcacctcacagaaatcacgatcctcacagtacaactaatagtcgagttcgctaaacggttacctggtttcgacaagctcttaagggaagatcagattgctcttctcaaggcctgttcaagtgaggtgatgatgttgaggatggccaggaagtacgacgctactacagactctatcctcttcgccaacaatcagccctataacagggagtcctatagcctagcaggcatgggcgatgtcattgaagacctgcttaggttttgtcgacatatgttcaacatgaaagtggacaatgctgaatacgctctccttacagctattgtcattttctcagagaggcctgcactaattgaaggctggaaggttgagaaaattcaagaaatatacttggaagctttgaagtcctacgtcgataacaggtcacgaccaagatcccctaccatattcgccaaactgttgtctgttttgacagaacttcgcacgctaggcaaccaaaattctgaaatgtgcttctcactcaagttgcaaaacaagaaattacccccattccttgcagaaatatgggacgtcaacacg

SEQ ID NO: 2 - DNA, 1257 bp, Artificial
  Nezara viridula clone pBK-CMVEcR11.1 (NvEcR11)
atgtcctccaacagctacgaaccctatagccccgcttcaaagctaggtcgagaggatctttcgccggcgaactctctgaacgggttctcggcggacagctgcgatggctccaagaagaagaaggggtcggcggcgcgacagcaggaggagctctgcctggtctgtggtgacagggcaagcggttaccattataacgcgctcacgtgcgaaggttgtaagggtttcttcagaaggagtataacgaagaacgccgtctatcaatgcaagtatggcaacaactgtgagatagacatgtacatgaggcgaaagtgtcaagagtgcaggctcaagaaatgtctgtctgttggcatgaggccggaatgtgttgtaccagagtaccaatgcgctgtgaaaagaaaagaaaagaaagcccagaaggacaaagataaaccagtctcgacgactaatggtagccctgaatcaataaaagtcgaagaaccacatagggttgtatataaaacagaaggtgaaactttgggccgaccagtcccaattaatggcgtcaaaccagtttcacccgaacaggaagaactcatccataggcttgtgtacttccagaacgagtacgagcacccttctgaggaagacctcagaaggattaatactccaaacgaggatgaagagcagagtgaccaaaggttcaggcacctcacagaaatcacgatcctcacagtacaactaatagtcgagttcgctaaacggttacctggtttcgacaagctcttaagggaagatcagattgctcttctcaaggcctgttcaagtgaggtgatgatgttgaggatggccaggaagtacgacgctactacagactctatcctcttcgccaacaatcagccctataacagggagtcctatagcctagcaggcatgggcgatgtcattgaagacctgcttaggttttgtcgacatatgttcaacatgaaagtggacaatgctgaatacgctctccttacagctattgtcattttctcagagaggcctgcactaattgaaggctggaaggttgagaaaattcaagaaatatacttggaagctttgaagtcctacgtcgataacaggtcacgaccaagatcccctaccatattcgccaaactgttgtctgttttgacagaacttcgcacgctaggcaaccaaaattctgaaatgtgcttctcactcaagttgcaaaacaagaaattacccccattccttgcagaaatatgggacgtcaacacg

SEQ ID NO: 3 - DNA, 1206 bp, Artificial
  Nezara viridula clone pBK-CMVUSP5.1 (NvUSP)
atggaagctaatgagcgaggactgagcttggagaacaacctgtcgctggtgggcccgcagtcgccgctggacatgaagccggacgcagccagcctcctcgcgggcagcttcagcccctcccaggcctccaaccctacaagccctgcgggatttggtatggcacataacagtgtacttggaaatggtaacaagagcctgaataccccttatcctcctaaccatccactgagtggttctaaacatctctgcagcatatgtggtgacagagcctcaggaaagcattatggagtttatagttgtgaaggttgtaaaggttttttcaaaagaacagtacgtaaagatttatcttatgcttgtcgtgaagataaacaatgtctggtagataaacgtcaaaggaatagatgccaatattgccgatatcagaaatgtttatcgatgggcatgaaaagggaagcagtccaggaagaaaggcaaagaactaaagagagagatcaaaatgaagttgaaagcaccagcagctttcacacagatatgcctgttgaaaggattttagaagcagaaagaagagttgatttcaaagtggagcctatggtagaatatgagaatgcaaatacactttttcaagcgactgataaacaactggtacagcttgttgaatgggctaaacaaataccacattttacctctctgcctatcgaagatcaagtactccttcttagagctggttggaatgagctactgatagcaggattttctcatcgctctattggtgttaaagaaaaaatagttttaggttctggagtaacagtttgtagaaataccgctcatcaagctggtgttgatacaatattcgacagagctctcactgaattagtttcaaaaatgagagaaatgaagatggataaagcagaacttggttgcctgagaactataatattatataatccagaagttcggggattgaggtcagtaggagaagttgaagctctgagggaaaaagtctatgcatcactcgaagaatatacaagatcaacccatccagaagagcctgggcgcttcgcgaaattgttacttcgactgccttctctgaggtctattggcctgaaatgccttgaaccactctttttctatagggttcttcatgacatacccatcgatacattcttattacagatgttggaatcatctgacttgtccaacagattg

SEQ ID NO: 4 - protein, 444 aa, Artificial
  Deduced amino acid sequence of NvEcR10
MCEESRRDEWTVGGGQNGCPSPTMSSNSYEPYSPASKLGREDLSPANSLNGFSADSCDGSKKKKGSAARQQEELCLVCGDRASGYHYNALTCEGCKGFFRRSITKNAVYQCKYGNNCEIDMYMRRKCQECRLKKCLSVGMRPECVVPEYQCAVKRKEKKAQKDKDKPVSTTNGSPESIKVEEPHRVVYKTEGETLGRPVPINGVKPVSPEQEELIHRLVYFQNEYEHPSEEDLRRIVSNTPNEDEEQSDQRFRHLTEITILTVQLIVEFAKRLPGFDKLLREDQIALLKACSSEVMMLRMARKYDATTDSILFANNQPYNRESYSLAGMGDVIEDLLRFCRHMFNMKVDNAEYALLTAIVIFSERPALIEGWKVEKIQEIYLEALKSYVDNRSRPRSPTIFAKLLSVLTELRTLGNQNSEMCFSLKLQNKKLPPFLAEIWDVNT

SEQ ID NO: 5 - protein, 419 aa, Artificial
  Deduced amino acid sequence of NvEcR11
MSSNSYEPYSPASKLGREDLSPANSLNGFSADSCDGSKKKKGSAARQQEELCLVCGDRASGYHYNALTCEGCKGFFRRSITKNAVYQCKYGNNCEIDMYMRRKCQECRLKKCLSVGMRPECVVPEYQCAVKRKEKKAQKDKDKPVSTTNGSPESIKVEEPHRVVYKTEGETLGRPVPINGVKPVSPEQEELIHRLVYFQNEYEHPSEEDLRRINTPNEDEEQSDQRFRHLTEITILTVQLIVEFAKRLPGFDKLLREDQIALLKACSSEVMMLRMARKYDATTDSILFANNQPYNRESYSLAGMGDVIEDLLRFCRHMFNMKVDNAEYALLTAIVIFSERPALIEGWKVEKIQEIYLEALKSYVDNRSRPRSPTIFAKLLSVLTELRTLGNQNSEMCFSLKLQNKKLPPFLAEIWDVNT

SEQ ID NO: 6 - protein, 402 aa, Artificial
  Deduced amino acid sequence of NvUSP
MEANERGLSLENNLSLVGPQSPLDMKPDAASLLAGSFSPSQASNPTSPAGFGMAHNSVLGNGNKSLNTPYPPNHPLSGSKHLCSICGDRASGKHYGVYSCEGCKGFFKRTVRKDLSYACREDKQCLVDKRQRNRCQYCRYQKCLSMGMKREAVQEERQRTKERDQNEVESTSSFHTDMPVERILEAERRVDFKVEPMVEYENANTLFQATDKQLVQLVEWAKQIPHFTSLPIEDQVLLLRAGWNELLIAGFSHRSIGVKEKIVLGSGVTVCRNTAHQAGVDTIFDRALTELVSKMREMKMDKAELGCLRTIILYNPEVRGLRSVGEVEALREKVYASLEEYTRSTHPEEPGRFAKLLLRLPSLRSIGLKCLEPLFFYRVLHDIPIDTFLLEMLESSDLSNRL

SEQ ID NO: 7 - protein, 16 aa, Artificial
  Tryptic digest peptide from NvEcR10
IVSNTPNEDEEQSDQR

SEQ ID NO: 8 - protein, 14 aa, Artificial
  Tryptic digest peptide from NvEcR11
INTPNEDEEQSDQR

SEQ ID NO: 9 - DNA, 138 bp, Artificial
  NvEcR screening probe
taccactataatccgctcacgtgcgaaggttgtaagggtttcttcagaaggagtataatgaagaacgccgtctatcaatgcaagtatggcaacaactgtgagatagacatgtacatgaggcgaaagtgtcaggagtgt

SEQ ID NO: 10 - protein, 46 aa, Artificial
  Peptide related to NvEcR screening probe
YHYNALTCEGCKGFFRRSIMKNAVYQCKYGNNCEIDMYMRRKCQEC

SEQ ID NO: 11 - DNA, 24 bp, Artificial
  Degenerate primer
  misc_feature (2)..(2): n is a or t
  misc_feature (3)..(3): n is c or g
  misc_feature (7)..(7): n is a or g
  misc_feature (10)..(10): n is a or g
  misc_feature (13)..(13): n is c or t
  misc_feature (16)..(16): n is c or t
  misc_feature (19)..(19): n is a or t
  misc_feature (22)..(22): n is c or t
cnntggnaancantanggngtnta

SEQ ID NO: 12 - DNA, 24 bp, Artificial
  Degenerate primer
  misc_feature (6)..(6): n is c or t
  misc_feature (12)..(12): n is c or t
  misc_feature (15)..(15): n is a or g
  misc_feature (18)..(18): n is a or g
  misc_feature (21)..(21): n is c or t
  misc_feature (24)..(24): n is a or g
catttntgatanctncantantgn

SEQ ID NO: 13 - DNA, 65 bp, Artificial
  Forward primer
gcggccgcatgggtatgagaggatcgcatcaccatcaccatcacaggccggaatgtgttgtacca

SEQ ID NO: 14 - protein, 19 aa, Artificial
  Peptide sequence related to forward primer sequence
MGMRGSHHHHHHRPECVVP

SEQ ID NO: 15 - DNA, 29 bp, Artificial
  Reverse primer
tctagagcttctcacgtgttgacgtccca

SEQ ID NO: 16 - DNA, 53 bp, Artificial
  Forward primer
ccatggcagactacaaggacgacgatgacaagaaaagggaagcagtccaggaa

SEQ ID NO: 17 - protein, 17 aa, Artificial
  Peptide sequence related to forward primer
MADYKDDDDKKREAVQE

SEQ ID NO: 18 - DNA, 29 bp, Artificial
  Reverse primer
ggtaccccaagctacaatctgttggacaa

SEQ ID NO: 19 - DNA, 435 bp, Artificial
  Cloned NvEcR10
  CDS (289)..(435)
gtggaggtagtgtcgccctgcgatctgtcgaacgttcactgtactcttcacttcttctgtcggagatcgttcgctattcactcattcaacgtggtgaacatctttcgtgtcggatttgtgttcagtgatcgcgggccggaacggagtttatataatattttattatttttgacagtgttggaaatttttggattaacccataagaactaggtcgaatggactcaacccattaccaaatcacaggtggaatcccgcagtttacagtctggatgtttacgatgatgtgagatgtgcgaggagagccggcgggacgagtggactgttgggggggggcagaacgggtgcccctccccgacaatgtcctccaacagctacgaaccctatagccccgcttcaaagctaggtcgagaggatctttcgccggcgaactctctg

SEQ ID NO: 20 - protein, 49 aa, Artificial
  Synthetic Construct
MCEESRRDEWTVGGGQNGCPSPTMSSNSYEPYSPASKLGREDLSPANSL

SEQ ID NO: 21 - DNA, 181 bp, Artificial
  Synthetic construct
  CDS (104)..(181)
ggaaagtgggccaactagtgaataggatgtcaatttcaaaatgttttgaatttttataataaatcattttctcaagatattttctttcgagttcaggaccaaaatggaagctaatgagcgaggactgagcttggagaacaacctgtcgctggtgggcccgcagtcgccgctggacatgaag

SEQ ID NO: 22 - protein, 26 aa, Artificial
  Synthetic Construct
MEANERGLSLENNLSLVGPQSPLDMK

SEQ ID NO: 23 - DNA, 584 bp, Artificial
  Synthetic construct
  CDS (1)..(129)
tcacccgaacaggaagaactcatccataggcttgtgtacttccagaacgagtacgagcacccttctgaggaagacctcagaaggattgtaagtgtaaggacaggtttttattattttttttcttattattaatctttcctctctattttaatataatcatttatataacatttgttgagtaaattttctactaacatcaaatttatttttaaaatgcatggattacacaatactccagagaatgtagtaaccatttaagctcagattaacttgcattagctattgaccaaatacccaaagttaatctccttgaaagcatgtctgcctttattagtttctcaatatttttcaaaaaacagaatttttccttttcatagcctcttaaattcaacccaacacctgttcctaatattgacctaaaatgggctcaatatagattactaaaagctgcatggttttttgtcataactttttgagaaaggattagggtgacagtcataaaaagactaaaatagtgaaaaaaaaattttattccaagtgtacgttttgacccaactttgcaggtctattttagggttagctag

SEQ ID NO: 24 - protein, 43 aa, Artificial
  Synthetic Construct
SPEQEELIHRLVYFQNEYEHPSEEDLRRIVSVRTGFYYFFSYY

SEQ ID NO: 25 - DNA, 233 bp, Artificial
  Synthetic construct
attttgagggagagtattataggaaaataaaggagggcaagccactcctatcagttacgttcccagtgcaaggttaatttgagtagattaaagtgaaatccatgcacttttatttagttttttctccttttcttttaaagctattccagccggaattaatttatattttattttcatttttacagaatactccaaacgaggatgaagagcagactgaccaaaggttcaggcaa

SEQ ID NO: 26 - protein, 39 aa, Artificial
  Sequence encoded by synthetic construct
FFLLFLLKLFQPELIYILFSFLQNTPNEDEEQSDQRFRQ